(19) United States
(12) Patent Application Publication
(10) Pub. No.: US 2024/0368247 A1
(43) Pub. Date: Nov. 7, 2024

(54) THERAPEUTIC APPLICATIONS OF TYPE 1 INSULIN-LIKE GROWTH FACTOR (IGF-1) RECEPTOR ANTAGONISTS

(71) Applicant: THE ROYAL INSTITUTION FOR THE ADVANCEMENT OF LEARNING/MCGILL UNIVERSITY, Montréal (CA)

(72) Inventor: Pnina BRODT, Côte St-Luc (CA)

(21) Appl. No.: 18/255,682

(22) PCT Filed: Dec. 1, 2021
(86) PCT No.: PCT/CA2021/051714

§ 371 Date: Jun. 2, 2023

Related U.S. Application Data
(60) Provisional application No. 63/120,442, filed on Dec. 2, 2020.

(51) Int. Cl. C07K 14/71; A61K 38/00; A61P 37/06; C07K 16/28

(52) U.S. Cl. CPC C07K 14/71; A61P 37/06; C07K 16/2878; A61K 38/00; C07K 2319/30

(57) ABSTRACT

The present disclosure concerns the reduction in the immunosuppression and the increase in the anti-tumor immune cytotoxicity in a tissue by using an antagonist of the type 1 insulin growth factor receptor (IGF-1R). The present disclosure also concerns the combination of the antagonist of the IGF-1R with an immune response activating agent to mitigate the symptom or treat a cancer in a subject.

### CROSS-REFERENCE TO RELATED APPLICATIONS

This application claims priority on U.S. provisional application Ser. No. 63/120,442 filed Dec. 2, 2020, the entire content of which is hereby incorporated by reference in its entirety.

### TECHNOLOGICAL FIELD

The present disclosure concern the use of an IGF-1 receptor antagonist, alone or in combination with an anti-cancer immune stimulating agent for reducing the immune suppression and increasing immune cell cytotoxicity in a microenvironment of a malignant tumor.

### BACKGROUND

Pancreatic ductal adenocarcinoma (PDAC) is currently the fourth leading cause of cancer-related deaths in the industrialized world. Despite remarkable advances in current diagnostic techniques, it has been widely reported that PDAC may surpass colorectal cancer to become the second leading cause of cancer-related death in the USA by 2030. There is a dearth of effective therapies for pancreatic cancer and the 5-year survival still stands at 5-9%, the lowest of any common malignancy, identifying this disease as an obvious “unmet need”. The most notable clinical features of PDAC are its propensity for aggressive local invasion, metastasis (mainly to the liver) and inherent resistance to conventional therapies. While approximately 50% of pancreatic cancer patients present with evidence of distant disease, particularly in the liver, the remaining patients have localized disease without detectable metastases. Of these, 15-20% of patients are operable and therefore potentially amenable to curative therapy, while ˜30% have locally advanced disease. Thus, therapies that can effectively reduce the incidence of pancreatic cancer metastases and/or metastatic outgrowth would have tremendous potential to significantly improve the therapeutic outcome such as survival.

It would thus be beneficial to be provided with a therapeutic agent which can be used to reduce the immunosuppression induced by a cancer and/or potentiate the use of an immune response activating agent.

### BRIEF SUMMARY

The present disclosure concerns the use of an antagonist of a type I insulin-like growth factor receptor to decrease immune suppression, increasing immune cytotoxicity and/or potentiate the therapeutic activity of an immune response activating agent. The antagonist of the type I insulin-like growth factor receptor can be used for alleviating a symptom or treating a cancer and/or an immune disease/disorder where a reduction of the immunosuppression is desirable.

According to a first aspect, the present disclosure provides a method for alleviating a symptom of, or treating a cancer in a subject in need thereof. Broadly, the method comprises administrating an effective amount of an antagonist of a type I insulin-like growth factor receptor (IGF-1R) prior to, concomitantly and/or after having administered an effective amount of an immune response activating agent to the subject so as to alleviate the symptom or treat the cancer. In an embodiment, the antagonist of the IGF-1R comprises a soluble IGF receptor. In another embodiment, the antagonist of the IGF-1R is chimeric protein comprising the soluble IGF-1 receptor as a first moiety and a human Fc as a second moiety. In still another embodiment, the antagonist of the IGF-1R comprises the amino acid sequence of SEQ ID NO: 6 or 8, a variant of the amino acid sequence of SEQ ID NO: 6 or 8 or a fragment of the amino acid sequence of SEQ ID NO: 6 or 8. In yet another embodiment, the immune response activating agent comprises an antagonistic antibody. In still a further embodiment, the antagonistic antibody is specific for an immune check-point. In specific embodiments, the anti-cancer immune stimulating agent comprises an anti-cytotoxic T-lymphocyte associated protein 4 (CTLA-4) antibody, an anti-programmed cell death 1 (PD-1) antibody, an anti-programmed cell death 1 ligand (PD-L1) antibody, an anti-programmed cell death 2 ligand (PD-L2) antibody, an anti-T cell immunoglobulin and mucin domain 3 (TIM-3) antibody and/or an anti-lymphocyte activation gene-3 (LAG-3) antibody. In still another embodiment, the immune response activating agent comprises an agonistic antibody. In yet a further embodiment, the immune response activating agent comprises an anti-TNF receptor superfamily member 9 (TNFRSF9) antibody, an anti-TNF receptor superfamily member 4 (TNFRSF4) antibody and/or an anti-TNF receptor superfamily member 18 (TNFRSF18) antibody. In another embodiment, the cancer is a carcinoma. In still a further embodiment, the cancer is pancreatic cancer. In yet another embodiment, the cancer is a metastatic cancer. In still a further embodiment, the metastatic cancer is a liver metastatic cancer. In yet a further embodiment, the subject is a mammalian subject, such as, for example, a human.

According to a second aspect, the present disclosure provides the use of an antagonist of a type I insulin-like growth factor receptor (IGF-1R) for alleviating a symptom of or treating a cancer in a subject in need thereof, as well as the use of an antagonist of a type I insulin-like growth factor receptor (IGF-1R) for the manufacture of a medicament alleviating a symptom of or treating a cancer in a subject in need thereof. The IGF-1R antagonist is used in combination with an immune response activating agent to the subject, so as to alleviate the symptom or treat the cancer. The IGF-1R is adapted to be used prior to, concomitantly and/or after the immune response activating agent. In an embodiment, the antagonist of the IGF-1R comprises a soluble IGF receptor. In another embodiment, the antagonist of the IGF-1R is chimeric protein comprising the soluble IGF-1 receptor as a first moiety and a human Fc as a second moiety. In still another embodiment, the antagonist of the IGF-1R comprises the amino acid sequence of SEQ ID NO: 6 or 8, a variant of the amino acid sequence of SEQ ID NO: 6 or 8 or a fragment of the amino acid sequence of SEQ ID NO: 6 or 8. In yet another embodiment, the immune response activating agent comprises an antagonistic antibody. In still a further embodiment, the antagonistic antibody is specific for an immune check-point. In specific embodiments, the anti-cancer immune stimulating agent comprises an anti-cytotoxic T-lymphocyte associated protein 4 (CTLA-4) antibody, an anti-programmed cell death 1 (PD-1) antibody, an anti-programmed cell death 1 ligand (PD-L1) antibody, an anti-programmed cell death 2 ligand (PD-L2) antibody, an anti-T cell immunoglobulin and mucin domain 3 (TIM-3) antibody and/or an anti-lymphocyte activation gene-3 (LAG-3) antibody. In still another embodiment, the immune response activating agent comprises an agonistic antibody. In yet a further embodiment, the immune response activating agent comprises an anti-TNF receptor superfamily member 9 (TNFRSF9) antibody, an anti-TNF receptor superfamily member 4 (TNFRSF4) antibody and/or an anti-TNF receptor superfamily member 18 (TNFRSF18) antibody. In another embodiment, the cancer is a carcinoma. In still a further embodiment, the cancer is pancreatic cancer. In yet another embodiment, the cancer is a metastatic cancer. In still a further embodiment, the metastatic cancer is a liver metastatic cancer. In yet a further embodiment, the subject is a mammalian subject, such as, for example, a human.

According to a third aspect, the present disclosure provides a method for alleviating a symptom of or treating a cancer in a subject in need thereof. Broadly, the method comprises administrating an effective amount of an immune response activating agent prior to, concomitantly and/or after having administered an effective amount of an antagonist of a type 1 insulin-like growth factor receptor (IGF-1R) to the subject so as to alleviate the symptom or treat the cancer. In an embodiment, the antagonist of the IGF-1R comprises a soluble IGF receptor. In another embodiment, the antagonist of the IGF-1R is chimeric protein comprising the soluble IGF-1 receptor as a first moiety and a human Fc as a second moiety. In still another embodiment, the antagonist of the IGF-1R comprises the amino acid sequence of SEQ ID NO: 6 or 8, a variant of the amino acid sequence of SEQ ID NO: 6 or 8 or a fragment of the amino acid sequence of SEQ ID NO: 6 or 8. In yet another embodiment, the immune response activating agent comprises an antagonistic antibody. In still a further embodiment, the antagonistic antibody is specific for an immune check-point. In specific embodiments, the anti-cancer immune stimulating agent comprises an anti-cytotoxic T-lymphocyte associated protein 4 (CTLA-4) antibody, an anti-programmed cell death 1 (PD-1) antibody, an anti-programmed cell death 1 ligand (PD-L1) antibody, an anti-programmed cell death 2 ligand (PD-L2) antibody, an anti-T cell immunoglobulin and mucin domain 3 (TIM-3) antibody and/or an anti-lymphocyte activation gene-3 (LAG-3) antibody. In still another embodiment, the immune response activating agent comprises an agonistic antibody. In yet a further embodiment, the immune response activating agent comprises an anti-TNF receptor superfamily member 9 (TNFRSF9) antibody, an anti-TNF receptor superfamily member 4 (TNFRSF4) antibody and/or an anti-TNF receptor superfamily member 18 (TNFRSF18) antibody. In another embodiment, the cancer is a carcinoma. In still a further embodiment, the cancer is pancreatic cancer. In yet another embodiment, the cancer is a metastatic cancer. In still a further embodiment, the metastatic cancer is a liver metastatic cancer. In yet a further embodiment, the subject is a mammalian subject, such as, for example, a human.

According to a fourth aspect, the present disclosure provides the use of an immune response activating agent for alleviating a symptom of or treating a cancer in a subject in need thereof as well as the use of an immune response activating agent for the manufacturing of a medicament for alleviating a symptom of or treating a cancer in a subject in need thereof. The immune response activating agent is used in combination with an antagonist of a type 1 insulin-like growth factor receptor (IGF-1R). The immune response activating agent is adapted to be administered prior to, concomitantly and/or after the IGF-1R. In an embodiment, the antagonist of the IGF-1R comprises a soluble IGF receptor. In another embodiment, the antagonist of the IGF-1R is chimeric protein comprising the soluble IGF-1 receptor as a first moiety and a human Fc as a second moiety. In still another embodiment, the antagonist of the IGF-1R comprises the amino acid sequence of SEQ ID NO: 6 or 8, a variant of the amino acid sequence of SEQ ID NO: 6 or 8 or a fragment of the amino acid sequence of SEQ ID NO: 6 or 8. In yet another embodiment, the immune response activating agent comprises an antagonistic antibody. In still a further embodiment, the antagonistic antibody is specific for an immune check-point. In specific embodiments, the anti-cancer immune stimulating agent comprises an anti-cytotoxic T-lymphocyte associated protein 4 (CTLA-4) antibody, an anti-programmed cell death 1 (PD-1) antibody, an anti-programmed cell death 1 ligand (PD-L1) antibody, an anti-programmed cell death 2 ligand (PD-L2) antibody, an anti-T cell immunoglobulin and mucin domain 3 (TIM-3) antibody and/or an anti-lymphocyte activation gene-3 (LAG-3) antibody. In still another embodiment, the immune response activating agent comprises an agonistic antibody. In yet a further embodiment, the immune response activating agent comprises an anti-TNF receptor superfamily member 9 (TNFRSF9) antibody, an anti-TNF receptor superfamily member 4 (TNFRSF4) antibody and/or an anti-TNF receptor superfamily member 18 (TNFRSF18) antibody. In another embodiment, the cancer is a carcinoma. In still a further embodiment, the cancer is pancreatic cancer. In yet another embodiment, the cancer is a metastatic cancer. In still a further embodiment, the metastatic cancer is a liver metastatic cancer. In yet a further embodiment, the subject is a mammalian subject, such as, for example, a human.

According to a fifth aspect, the present disclosure concerns a method of reducing the immune suppression and increasing the immune cytotoxicity in a tissue in need thereof. Broadly, the method comprises contacting an antagonist of a type 1 insulin growth factor receptor (IGF-1R) with the tissue so as to reduce the immune suppression and increase the immune cytotoxicity when compared to a control tissue that was not contacted with the antagonist of the IGF-1R. In an embodiment, the method is for reducing myeloid derived suppressor cells, immunosuppressive (N2) neutrophils and/or anti-inflammatory immunosuppressive (M2) tumor-associate macrophages in the tissue. In a further embodiment, the method is for increasing and activating dendritic cells and/or increasing pro-inflammatory (N1) neutrophils in the tissue. In an embodiment, the tissue comprises a malignant tumor. In a further embodiment, a micro-environment exhibiting a state of immune suppression is present in the vicinity of the malignant tumor. In a further embodiment, the malignant tumor is from a carcinoma, a melanoma or a glioma. In yet another embodiment, the malignant tumor is a metastasis. In still a further embodiment, the malignant tumor is from a pancreatic carcinoma, such as, for example, from a liver metastasis. In an embodiment, the method is for decreasing the activation of hepatic stellate cells in the tumor microenvironment of the liver metastasis. In still another embodiment, the method is for increasing, in the tissue, when compared to the control tissue: CD11b+, CD11c+ and MHCII+ immune-accessory cells; CD11c+ and MHCII+ immune response cells; ICAM-1+ immune cells; CD4+ immune cells; CD8+ cells; and/or CD68+ cells. In a specific embodiment, the CD8+ or CD4+ cells are also PD1+ cells. In still a further embodiments, the method is for decreasing, in the tissue, when compared to the control tissue: CD11b+, Ly6G+ and Ly6C+ immunosuppressive cells; CD163+ immune cells; and/or CD206+ immune cells. In still a further embodiment, the method is for decreasing, in the tissue, when compared to the control tissue: TGF-β; collagen I; and/or α-smooth muscle actin expressing cells. In still another embodiment, the method is for increasing, in the tissue when compared to a control tissue: IFN-γ; and/or granzyme B. In an embodiment, the malignant tumor is present in a subject. In another embodiment, the subject is a mammalian subject, such as, for example, a human.

According to a sixth aspect, the present disclosure concerns the use of an antagonist of a type 1 insulin growth factor receptor (IGF-1R) for reducing the immune suppression and increasing the immune cytotoxicity in a tissue as well as the use of an antagonist of a type 1 insulin growth factor receptor (IGF-1R) for the manufacture of a medicament for reducing the immune suppression and increasing the immune cytotoxicity in a tissue. The reduction in immune suppression and the increase in immune cytotoxicity is observed when compared to a control tissue that was not contacted with the antagonist of the IGF-1R. In an embodiment, the use is for reducing myeloid derived suppressor cells, immunosuppressive (N2) neutrophils and/or anti-inflammatory immunosuppressive (M2) tumor-associate macrophages in the tissue. In a further embodiment, the use is for increasing and activating dendritic cells and/or increasing pro-inflammatory (N1) neutrophils in the tissue. In an embodiment, the tissue comprises a malignant tumor. In a further embodiment, a micro-environment exhibiting a state of immune suppression is present in the vicinity of the malignant tumor. In a further embodiment, the malignant tumor is from a carcinoma, a melanoma or a glioma. In yet another embodiment, the malignant tumor is a metastasis. In still a further embodiment, the malignant tumor is from a pancreatic carcinoma, such as, for example, from a liver metastasis. In an embodiment, the use is for decreasing the activation of hepatic stellate cells in the tumor microenvironment of the liver metastasis. In still another embodiment, the use is for increasing, in the tissue, when compared to the control tissue: CD11b+, CD11c+ and MHCII+ immune-accessory cells; CD11c+ and MHCII+ immune response cells; ICAM-1+ immune cells; CD4+ immune cells; CD8+ cells; and/or CD68+ cells. In a specific embodiment, the CD8+ or CD4+ cells are also PD1+ cells. In still a further embodiments, the use is for decreasing, in the tissue, when compared to the control tissue: CD11b+, Ly6G+ and Ly6C+ immunosuppressive cells; CD163+ immune cells; and/or CD206+ immune cells. In still a further embodiment, the use is for decreasing, in the tissue, when compared to the control tissue: TGF-β; collagen I; and/or α-smooth muscle actin expressing cells. In still another embodiment, the method is for increasing, in the tissue when compared to a control tissue: IFN-γ; and/or granzyme B. In an embodiment, the malignant tumor is present in a subject. In another embodiment, the subject is a mammalian subject, such as, for example, a human.

### BRIEF DESCRIPTION OF THE DRAWINGS

Having thus generally described the nature of the invention, reference will now be made to the accompanying drawings, showing by way of illustration, a preferred embodiment thereof, and in which:

FIGS. 1A to 1D illustrate that the IGF-Trap preferentially inhibits the growth of liver metastases in an orthotopic PDAC model. LMP cells (5×10⁵ in Matrigel) were implanted in the pancreas of immunocompetent, histocompatible B6/129F1 (B129) male mice. Treatment with 5 mg/kg IGF-Trap (or PBS) was initiated 1 (IGF-Trap (Day1)) or 3 (IGF-Trap (Day 3)) days later and continued on alternate days for a total of 5 injections per mouse. Animals were euthanized 3 weeks post tumor implantation, local pancreatic tumors measured and visible metastases on the surface of the liver enumerated. Results are based on 4 independent experiments. FIG. 1A shows the numbers of metastases per liver and (on top) the incidence of hepatic metastases per group. FIG. 1B shows the average sizes of the metastases expressed as means (±SD) in each group. FIG. 1C shows representative hematoxylin and eosin (H&E) stained PPFE sections of livers from each of the treatment groups. FIG. 1D shows the volumes of individual local pancreatic tumors of the same mice calculated using the formula ½ (length×width²) with 2 representative tumors from each of the treatment groups shown on top. Box and whiskers graphs: the box extends from the 25^{th} to 75^{th} percentiles, the middle line denotes de median and the whiskers extends from the minimum to the maximum value. *p≤0.05; **P<0.01; ****P<0.0001; NS—not significant.

FIGS. 2A and 2B illustrate that the IGF-Trap treatment alters the tumor immune microenvironment (TIME) in the liver. Immune-profiling with the NanoString Geomax Profiler was performed on FFPE liver sections that were obtained from mice inoculated via the intrasplenic/portal route with 5×10⁵ LMP cells 21 days earlier and treated with a total of five tail vein injections of 5 mg/kg IGF-Trap or PBS (control) on alternate day. FIG. 2A shows representative regions of interest (ROI) selected based on the size of the metastases to depict early (n=3) or late (n=3) immune cell recruitment events. A more detailed image of the ROI selected for analysis can be seen in FIG. 8A hereinbelow. FIG. 2B shows a Heat map generated based on changes in the expression of immune cell surface markers expressed as log 10 (fold change) in expression relative to normal (tumor-free) liver that was used as baseline. Additional information on changes in specific cell surface markers can be seen in FIG. 8B herein below.

FIGS. 3A to 3F illustrate that the IGF-Trap reduces the accumulation of immunosuppressive cells in PDAC liver metastases. Liver immune cells were isolated 14 days post intrasplenic/portal injection of 1×10⁵ LMP cells and five i.v. injections of 5 mg/kg IGF-Trap or PBS on alternate days, and immunostained with the indicated antibodies. In a separate experiment, the same treatment protocol was used, and cryostat sections prepared for analysis by immunohistochemistry (IHC). FIG. 3A shows, on top, representative flow cytometric contour plots obtained with each of the indicated immune cell populations that were first gated for size, viability, and CD45 expression (for detailed gating strategy, see FIG. 10 hereinbelow). It also shows, bottom, bar graphs as mean proportions (±SEM) of MDSC, G-MDSC, and Mo-MDSC per liver based on 4 mice per group, analyzed individually. FIG. 3B shows representative flow cytometric contour plots of activated dendritic cells identified based on expression CD11c and MHCII (left). It also shows bar graphs of the mean proportions (±SEM) of CD11b+MHCII+ per liver based on 4 mice per group, analyzed individually (right). FIG. 3C shows representative contour plots obtained for CD11b+Ly6G^{high}Ly6C^{low} cells expressing ICAM-1 (left). It also shows bar graphs of the mean proportions (±SEM) of Ly6G+ICAM-1+ cells (markers of N1 neutrophils) in each group based on analysis of these mice (right). FIG. 3D shows (top) representative confocal images of 10 μm cryostat liver sections immunostained with the indicated antibodies followed by Alexa Fluor 568 (green) for CD11c, Alexa Fluor 647 for MHCII (red), and DAPI (blue). Shown on the right of the confocal images are the mean numbers (±SEM) of the indicated cells per field counted in 15-20 fields per section (n=2 or 3) derived from 3 mice per group. It also shows (bottom) representative confocal images obtained with the indicated antibodies followed by Alexa Fluor 568 (red) for F4/80, Alexa Fluor 647 (yellow) for CD206, and DAPI (blue). Shown on the right of the confocal images are the means (±SEM) of the indicated cells counted as in (D-top). MDSC were functionally characterized using a T cell proliferation assay. FIG. 3E shows the results of a separate experiment where mice were treated as described for (FIGS. 3A-3C) and FACS sorting used to isolate CD11b+Ly6G+Ly6C+ cells from IGF-Trap treated and control mice. Cells were then incubated with CFSE-labeled activated T cells and T cell proliferation analyzed by flow cytometry. Results are shown as a representative flow cytometry profile (left) and in the bar graph (right) the mean proportions (±SEM) of dividing CD3+ T cells based on triplicate samples. Neutrophils were functionally characterized based on mRNA expression profiles. FIG. 3F shows the results of qPCR analysis performed on RNA extracted from FACS sorted neutrophils (left). Results in the bar graph (right) are based on 3 separate analyses and expressed as means (±SD) expression levels (normalized to GAPDH) relative to untreated mice that were assigned a value of 1. Scale bar (D)—100 μm; *p≤0.05; **P<0.01; ***P<0.001; ****P<0.0001.

FIGS. 4A to 4E illustrate that the IGF-Trap treatment enhances T cell recruitment and potentiates their function in the tumor microenvironment (TME). B6129 F1 mice were injected via the intrasplenic/portal route with 1×10⁵ LMP cells, treated with 5 mg/kg IGF-Trap or PBS 1, 4, 7, and 10 days post tumor inoculation and sacrificed on day 14. FIG. 4A are representative confocal images of 10 μm cryostat liver sections immunostained with the indicated antibodies and Alexa Fluor 568 (red) for CD8 (A-top), Alexa Fluor 568 (red) for CD4 (A-bottom) and DAPI (blue). Shown in the bar graphs (right) are the mean numbers (±SEM) of the indicated cells per field counted in 15-20 fields per section derived from 3 mice per group. Scale bar—100 μm; *p≤0.05; **P<0.01; ***P<0.001. FIG. 4B shows the results of flow cytometry (left) performed on immune cells which isolated from mice treated as described in FIG. 4A and stimulated for 4 h with PMA and ionomycin in the presence of a protein transport inhibitor. It also provides a bar graph (right) as the mean proportions of CD8+IFN-γ+ cells per liver (±SEM) based on 3 livers per group analyzed individually. Scale bar—100 μm; *p≤0.05; **P<0.01; ***P<0.001. FIG. 4C shows results of qPCR (±SEM) performed on RNA extracted from CD3+CD8+ T cells that were FACS sorted from livers of tumor-injected mice treated as in FIG. 4A (data normalized to GAPDH; n=3). Scale bar—100 μm; *p≤0.05; **P<0.01; ***P<0.001. FIG. 4D shows, on the left, representative confocal images of cells immunostained with antibodies to Ki-67 (green) and with DAPI (blue) and, on the right, the mean numbers (±SEM) of Ki67+ cells per field based on 15-20 sections obtained from 3 animals per group. Scale bar—100 μm; *p≤0.05; **P<0.01; ***P<0.001. FIG. 4E shows the effect of IGF-Trap on hepatic stellate cell activation analyzed in B6129-Col-GFP mice. Mice were injected via the intrasplenic/portal route with 1×10⁵ LMP cells, treated with 5 mg/kg IGF-Trap or PBS on days 2 and 4 post tumor inoculation and sacrificed on day 7. Activated HSC recruited into tumor-infiltrated areas and identified based on type I collagen production and α-SMA expression were quantified. Shown in (left) are representative confocal images of 10 μm cryostat liver sections immunostained with antibodies to α-SMA followed by Alexa Fluor 568 secondary antibody (yellow), DAPI-stained (blue) and expressing Col-GFP (green). Shown in the bar graph (right) are the mean numbers of activated HSCs per field (±SEM) based on 15-20 sections obtained from 3 animals per group. Scale bar—50 μm; *p≤0.05; **P<0.01; ***P<0.001.

FIGS. 5A to 5H illustrate that the IGF-Trap inhibits the growth of pancreatic carcinoma liver metastases. Experimental liver metastases were generated by inoculation of 1×10⁵ (for males—FIGS. 5A-5D) or 5×10⁵ (for females—FIGS. 5E-5H) LMP cells via the intrasplenic/portal route. Treatment with 5 mg/kg IGF-Trap was initiated 1 day later and continued twice weekly for a total of 5 injections per mouse. Mice were sacrificed 21 days post tumor inoculation and visible metastases on the surface of the liver enumerated prior to fixation. Results are based on 2 experiments each for male and female mice. Scale bar corresponds to 100 μm. Box and whiskers graphs: the box extends from the 25th to 75th percentiles, the middle line denotes de median and the whiskers extends from the minimum to the maximum value. T-tumor; L-liver; *p<0.05; **p<0.01. FIG. 5A shows the numbers of metastases per each liver in the male mice. FIG. 5B shows representative livers of male mice from each group where arrows denote visible metastases. FIG. 5C shows the mean diameters of metastases per liver of male mice treated. FIG. 5D shows images of H&E stained FFPE liver sections (n=9) of male mice. FIG. 5E shows the numbers of metastases per each liver in the female mice. FIG. 5F shows representative livers of female mice from each group where arrows denote visible metastases. FIG. 5G shows the mean diameters of metastases per liver of female mice treated. FIG. 5H shows images of H&E stained FFPE liver sections (n=9) of female mice.

FIGS. 6A to 6M illustrate that the IGF-Trap enhances the anti-metastatic effect of immunotherapy. Experimental liver metastases were generated by the inoculation of 1×10⁵ (for males, FIGS. 6A-E), and 5×10⁵ (for females—FIGS. 6F-J) LMP cells via the intrasplenic/portal route. Treatment with 5 mg/kg IGF-Trap (or PBS) i.v. was initiated 1 day post tumor inoculation and continued twice weekly for a total of 5 injections per mouse. Treatment with 10 mg/kg PD-1 or the IgG isotype control was administered i.p. on days 2, 5, and 8 post tumor inoculation. Mice were sacrificed on day 21 and visible liver metastases enumerated without prior fixation. Results are based on 2 experiments each, for male and female mice. Box and whiskers graphs: the box extends from the 25^{th} to 75^{th} percentiles, the middle line denotes de median and the whiskers extends from the minimum to the maximum value. *p≤0.05; **P<0.01; ***P<0.001; ****P<0.0001; NS—not significant. FIG. 6A shows the numbers of metastases counted per liver of male mice. FIG. 6B shows mean diameters of the visible metastases per liver of male mice. There were no significant differences in either the numbers or sizes of metastases between untreated mice and mice treated with the IgG isotype control. FIG. 6C shows representative livers from male mice in each group where arrows denote metastases. FIG. 6D shows representative images of H&E stained FFPE liver sections of male mice where metastases are encircled. FIG. 6E shows means of total tumor surface area per section (±SEM) expressed as % of total liver surface area and based on analysis of 9 sections per group for male mice. Scale bar is 5 mm. FIG. 6F shows the numbers of metastases counted per liver of female mice. FIG. 6G shows mean diameters of the visible metastases per liver of female mice. There were no significant differences in either the numbers or sizes of metastases between untreated mice and mice treated with the IgG isotype control. FIG. 6H shows representative livers from female mice in each group where arrows denote metastases. FIG. 6I shows representative images of H&E stained FFPE liver sections of female mice where metastases are encircled. FIG. 6J shows means of total tumor surface area per section (±SEM) expressed as % of total liver surface area and based on analysis of 9 sections per group for female mice. Scale bar is 5 mm. FIG. 6K illustrates the effect of combinatorial treatment was also tested in C57BI/6 male mice injected with 2.5×10⁵ FC1199 cells via the intrasplenic/portal route and treated as described for LMP injected mice. This figures shows the numbers of metastases counted per liver. FIG. 6L shows the mean tumor diameter of the visible metastases C57BI/6 male mice injected with 2.5×10⁵ FC1199 cells via the intrasplenic/portal route and treated as described for LMP injected mice. FIG. 6M shows representative livers of each group of C57BI/6 male mice injected with 2.5×10⁵ FC1199 cells via the intrasplenic/portal route and treated as described for LMP injected mice. Arrows denote metastases.

FIGS. 7A to 7E illustrate the increased T cell recruitment and decreased PD-1 expression in the liver TME following combinatorial therapy. Liver immune cells were isolated 14 days post intrasplenic/portal injection of 1×10⁵ LMP cells and the treatment protocol described in the legend to FIGS. 6A to 6M and analyzed by flow cytometry. FIG. 7A shows, on the left. representative flow cytometric contour plots obtained with each of the indicated immune cells populations that were first gated as outlined in FIG. 10 hereinbelow. FIG. 7A also provides, on the right, bar graphs of the mean proportions (%) (±SEM) of MDSC per liver based on 3 mice per group, analyzed individually. FIG. 7B shows the mean numbers of the indicated cells obtained per liver (±SEM) expressed as a ratio to untreated mice that were assigned a value of 1 and based on 3 mice per group, analyzed individually. FIG. 7C shows, on the left, representative contour plots obtained for CD8+ cells immunostained with antibodies to the indicated immune checkpoints. FIG. 7C shows, on the right, bar graphs the relative proportions (%) of positive T cells (±SEM) based on the analyses of 3 livers per group. FIG. 7D shows, on top, representative confocal images of 10 μm cryostat liver sections immunostained with the indicated antibodies followed by Alexa Fluor 568 (red) for CD8, Alexa Fluor 647 for PD-1 (green), and DAPI (blue). FIG. 7D shows, bottom, the mean numbers (±SEM) of the CD8+ T cells per field (expressing or not PD-1), counted in 15-20 fields per section derived from 3 mice per group. Note that the number of CD8+ T cells increased significantly in both IGF-Trap and combination therapy-treated mice but the ratio of PD-1+:CD8+ cell decreased significantly only in mice that received the combination therapy. FIG. 7E shows the results of flow cytometry (left) performed on immune cells which were isolated from mice treated as in (FIG. 7A) and stimulated for 4 h as described in the legend to FIG. 4 A to 4E. In the bar graph (right) are the mean proportions of CD8+IFN-γ+ cells per liver (±SEM) based on 3 livers per group, analyzed individually. Scale bar—100 μm; *p≤0.05; **P<0.01; ***P<0.001; ****P<0.0001; NS—not significant.

FIGS. 8A and 8B illustrate that IGF-Trap treatment alters the tumor immune microenvironment in the liver. Immune-profiling with the NanoString Geomax Profiler was performed on FFPE liver sections that were obtained from mice inoculated with 5×10⁵ LMP cells via the intrasplenic/portal route, treated with a total of five tail vein injections of 5 mg/kg IGF-Trap or saline (Vh) each, on alternate day and sacrificed on day 21. FIG. 8A shows the regions of interest (ROI) within the FFPE liver sections selected for profiling. FIG. 8B shows the summaries of the changes in the signal levels of the indicated markers associated with early (left) and late (right) events in immune cell recruitment as compared to signal intensity in “tumor-free” areas within the same sections that were assigned a value of 1.

FIGS. 9A and 9B illustrate reduced IGF-IR expression and activation levels in immune cells following IGF-Trap treatment. B6129 F1 mice were injected via the intrasplenic/portal route with 1×10⁵ LMP cells, treated with 5 mg/kg IGF-Trap or saline (Vh) 1, 4, 7, and 10 days post tumor inoculation and sacrificed on day 14. FIG. 9A shows representative confocal images of 10 μm cryostat liver sections immunostained with the indicated antibodies followed by Alexa Fluor 568 for CD11b (left panels), Alexa Fluor 647 for IGF-1R (middle panels) and DAPI (right panels). Shown in the bar graph (right) are the mean numbers (±SE) of double positive cells per field based on quantification of 10-12 fields in sections derived from 3 mice per group. Scale bar 100 μm; ****P<0.0001. FIG. 9B shows representative confocal images of sections stained with Alexa Fluor 568 (red) for CD11b, Alexa Fluor 647 (green) for pIGF-1R and DAPI (blue). Shown in the bar graph (right) are the mean numbers (±SE) of double positive cells per field based on quantification of 10-12 fields in sections derived from 3 mice per group. Scale bar 100 μm; ****P<0.0001.

FIG. 10 illustrates the flow cytometry gating strategy to obtain the results presented in FIGS. 3A to 3F and 7A to 7E. The complete flow cytometric gating strategy for immunophenotyping and sorting hepatic MDSC, DC, and T cells is shown.

FIG. 11 shows that PDAC LMP and FC1199 cells express PD-L1. RNA was extracted from the PDAC cells using TRIzol and cDNA was synthesized from the isolated RNA using the High Capacity cDNA Reverse Transcription Kit. Shown are results of a PCR analysis performed using the PD-L1 (CD274) primer set.

FIG. 12 illustrates the prolonged survival of mice treated with the combinatorial IGF-Trap/anti PD-1 therapy.

### DETAILED DESCRIPTION

The present disclosure concerns the use of an antagonist of the type I insulin-like receptor 1 (IGF-1R) to mitigate immunosuppression (and in some embodiments localized immunosuppression) and favor a pro-inflammatory immune response. Immunosuppression can be observed in various diseases or conditions such as cancers, endocrine disorders (such as acromegaly), diabetes, thyroid eye diseases, skin diseases (such as acne and psoriasis), immune disorders where a reduction of the immunosuppression is desirable as well as frailty. In some embodiments, the antagonist of the IGF-1R of the present disclosure can be used to mitigate the immunosuppression present in the tumor micro-environment (TME) alone or in combination with immune response activating agents such as, for example, immune checkpoint inhibitors. Recent advances in targeting immune checkpoints such as PD-1 and CTLA-4 have yielded promising therapeutic results in several aggressive and treatment-refractory cancers such as malignant melanoma, small cell lung cancer and renal cell carcinoma. However, to date, immunotherapy has failed to show promise in the treatment of PDAC. This, despite compelling clinical evidence for an effector T cell infiltrate in PDAC, that, when present, has positive prognostic implications and can become reactive to the autologous tumors when further potentiated ex vivo. This failure of immunotherapy to alter the course of PDAC may be due, at least in part, to the presence of immunosuppressive cells such as myeloid derived suppressor cells (MDSC) and tumor-associated macrophages (TAM) that polarize to an immunosuppressive M2 phenotype in the tumor microenvironment (TME) and impede T cell mediated cytotoxicity, as well as promote PDAC progression through the release of pro-angiogenic and pro-invasive factors such as VEGF and matrix metalloproteinase (MMPs). Thus, therapeutic approaches that can co-target the immunosuppressive TME of PDAC, while also reversing T cell exhaustion could enhance the efficacy of immunotherapy and are currently being sought.

### Antagonists of the Type I Insulin-Like Growth Factor Receptor

The receptor for the type I insulin-like growth factor (IGF-IR) has been identified as a target for anti-cancer therapy. IGF-IR is a heterotetrameric receptor tyrosine kinase (RTK) consisting of two 130-135 kDa α and two 90-95 kDa β chains, with several α-α and α-β disulfide bridges. It is synthesized as a polypeptide chain of 1367 amino acids that is glycosylated and proteolytically cleaved into α- and β-subunits that multimerize to form a tetramer. The ligand binding domain is on the extracellular α subunit, while the β subunit consists of an extracellular portion linked to the α subunit through disulfide bonds, a transmembrane domain and a cytoplasmic portion with a kinase domain and several critical tyrosines and serine involved in transmission of ligand-induced signals.

The ability of cancer cells to detach from the primary tumor and establish metastases in secondary organ sites remains the greatest challenge to the management of malignant disease. The liver is a major site of metastasis for some of the most prevalent human malignancies, particularly carcinomas of the upper and lower gastrointestinal (GI) tract. IGF-IR expression and function are critical for liver metastases formation in different tumor types. Tumor cells engineered to express a soluble form of IGF-IR (sIGFIR) lost the ability to metastasize to the liver.

An effective strategy for blocking the action of cellular receptor tyrosine kinases (RTKs), like the IGF-1R, is the use of soluble variants of these receptors that can bind and reduce ligand bioavailability to the cognate receptor in a highly specific manner. Such soluble variants of cellular receptor tyrosine kinases that bind and reduce ligand bioavailability to the cognate receptor in a highly specific manner are referred to herein as “decoy” receptors or “Trap” proteins (because they “trap” the ligand). The terms “decoy receptor”, “Trap protein” (or simply “Trap”) and “soluble receptor” are used interchangeably herein.

The present disclosure concerns the use of one or more antagonist of the type I insulin-like growth factor receptor (IGF-1R), alone or in combination with an immune response activating agent, for mitigating or reducing immune suppression. The antagonist of the IGF-1R is understood to limit the biological activity of the IGF-1R by binding to the IGF-1R ligand(s) and preventing same from binding to the IGF-1R. The antagonist of the IGF-1R thus acts like a biological sink, trap or decoy for the ligand(s) of the IGF-1R. IGF-1R traps are known in the art and have been described, for example, in U.S. Pat. Nos. 6,084,085 and 10,538,575 both incorporated herewith in their entirety.

The antagonist of the type 1 insulin growth factor receptor (IGF-1R) described herein has affinity towards the insulin-like growth factor 1 (IGF-I) and insulin-like growth factor receptor 2 (IGF-II). In some cases, the affinity of the antagonist of the IGF-1R for IGF-II may be unexpectedly about the same as its affinity for IGF-I. In some cases, the antagonist of the IGF-1R may unexpectedly have higher affinity for IGF-II than IGF-I. In some cases, the antagonist of the IGF-1R may have higher affinity for IGF-I than IGF-II. In some embodiments, the antagonist of the IGF-1R binds with high specificity to IGF-I and IGF-II as compared to insulin. For example, as determined using surface plasmon resonance, the affinity of the antagonist of the IGF-1R is about 1-2000 fold lower for insulin than for the IGF-I and IGF-II ligands. In some embodiments, the term “about the same” as in, e.g., “about the same binding affinity”, refers to two values that are approximately the same within the limits of error of experimental measurement or determination. For example, two values which are about 5%, about 10%, about 15%, about 20%, about 25%, or about 30% apart from each other, after correcting for standard error, are considered to be “about the same”. Two values that are “about the same” may also be referred to as “similar” herein, as in, e.g., two proteins having similar binding affinity. In one embodiment, “about the same” or “similar” binding affinity refers to binding affinities where one affinity is not more than 2- or 3-fold greater than the other. In another embodiment, a difference in binding affinity of at least about 6-fold or at least about 10-fold means that the two binding affinities are not “about the same” or “similar”.

In some embodiments, the antagonist of the IGF-1R has an in vivo stability (half-life) in mice of between 35 and 48 hours, which would be expected to provide a half-life in humans that is amply sufficient for therapeutic applications.

In an embodiment, the antagonist of the IGF-1R has the amino acid sequence of SEQ ID NO: 1, 2 or 3, is a variant of the amino acid sequence of SEQ ID NO: 1, 2 or 3 or is a fragment of the amino acid sequence of SEQ ID NO: 1, 2 or 3.

In an embodiment, the antagonist of the IGF-1R is a chimeric polypeptide comprising a first moiety which acts as a sink for the IGF-1R ligands (e.g., the antagonist of the IGF-1R) and a second carrier moiety. In an embodiment, the first moiety has the amino acid sequence of SEQ ID NO: 1, 2 or 3, is a variant of the amino acid sequence of SEQ ID NO: 1, 2 or 3 or is a fragment of the amino acid sequence of SEQ ID NO: 1, 2 or 3.

In some embodiments, the second carrier moiety comprises the Fc domain of an antibody, such as, for example, the Fc domain of an human antibody (e.g, the IgG1 human antibody). In some embodiments, it may be necessary to modify the second carrier moiety, such as the Fc moiety, so as to the limit the oligomerization of chimeric polypeptide (e.g., the creation of high molecular weight (HMW) aggregates during storage) of the chimeric polypeptide. For example, in some modified Fc domains, cysteines in the hinge region of the Fc domain were replaced with serine residues. In other modified Fc domains, a 11-amino acid linker was replaced with a 22-amino acid flexible (GS) linker. In some modified Fc domains, both of these approaches (mutation of Fc hinge Cys residues, and utilization of a longer flexible linker) were combined. In further modified Fc domains, the Fc hinge region was truncated to retain only the lower Cys residue and the length of the flexible linker was increased to 27 or 37 amino acids. In an embodiments, the Fc domain moiety may have the advantage of being sufficiently long and flexible to allow not only binding to the FcRn receptor for improved pharmacokinetic properties (half-life), but also to allow simultaneous binding of the Fc portions to the FcRγIII receptor ectodomain that may confer other beneficial properties (e.g., complement function). Modified Fc domain have been described in U.S. Pat. No. 10,538,575 incorporated herein in its entirety.

As used herein, the term “immunoglobulin heavy chain constant region” is used interchangeably with the terms “fragment crystallizable region”, “Fc”, “Fc region” and “Fc domain” and is understood to mean the carboxyl-terminal portion of an immunoglobulin heavy chain constant region, a variant or fragment thereof capable of binding a Fc receptor. As is known in the art, each immunoglobulin heavy chain constant region comprises four or five domains. The domains are named sequentially as follows: CH1-hinge-CH2-CH3(-CH4). CH4 is present in IgM, which has no hinge region. The immunoglobulin heavy chain constant region useful in the fusion proteins of the invention may comprise an immunoglobulin hinge region, a CH2 domain and a CH3 domain. As used herein, the term immunoglobulin “hinge region” is understood to mean an entire immunoglobulin hinge region or at least a portion of the immunoglobulin hinge region sufficient to form one or more disulfide bonds with a second immunoglobulin hinge region.

As used herein, in some embodiments “Fc” includes modified Fc domains, e.g., Fc domains which are modified to remove one or more Cys residues, e.g., to replace one or more Cys residues with Ser residues. In an embodiment, fusion proteins having modified Fc domains do not produce high molecular weight (HMW) species or produce a reduced amount of HMW species compared to fusion proteins having unmodified Fc domains.

It is contemplated that suitable immunoglobulin heavy chain constant regions may be derived from antibodies belonging to each of the immunoglobulin classes referred to as IgA, IgD, IgE, IgG, and IgM, however, immunoglobulin heavy chain constant regions from the IgG class are preferred. Furthermore, it is contemplated that immunoglobulin heavy chain constant regions may be derived from any of the IgG antibody subclasses referred to in the art as IgG1, IgG2, IgG3, and IgG4. In one embodiment, an Fc region is derived from IgG1. In another embodiment, an Fc region is derived from IgG2. In yet another embodiment, the Fc region is derived from a human immunoglobulin region, such as, for example, a human IgG1.

Immunoglobulin heavy chain constant region domains have cross-homology among the immunoglobulin classes. For example, the CH2 domain of IgG is homologous to the CH2 domain of IgA and IgD, and to the CH3 domain of IgM and IgE. Preferred immunoglobulin heavy chain constant regions include protein domains corresponding to a CH2 region and a CH3 region of IgG, or functional portions or derivatives thereof. The choice of particular immunoglobulin heavy chain constant region sequences from certain immunoglobulin classes and subclasses to achieve a particular result is considered to be within the level of skill in the art. The Fc regions of the present invention may include the constant region such as, for example, an IgG-Fc, IgG-C_{H}, an Fc or C_{H} domain from another Ig class, i.e., IgM, IgA, IgE, IgD or a light chain constant domain. Truncations and amino acid variants or substitutions of these domains may also be included.

In an embodiment, the Fc moiety of the chimeric polypeptide has the amino acid sequence of SEQ ID NO: 13, a variant thereof or a fragment thereof. In an embodiment, the Fc moiety of the chimeric polypeptide has the amino acid sequence of SEQ ID NO: 14, a variant thereof or a fragment thereof. In an embodiment, the Fc moiety of the chimeric polypeptide has the amino acid sequence of SEQ ID NO: 15, a variant thereof or a fragment thereof. In an embodiment, the Fc moiety of the chimeric polypeptide has the amino acid sequence of SEQ ID NO: 16, a variant thereof or a fragment thereof.

A variety of nucleic acid molecules encoding the chimeric polypeptide may also be used to make the antagonist of the IGF-1R of the present disclosure. For example, the nucleic acid molecules may encode in a 5′ to 3′ direction, either the immunoglobulin heavy chain constant region and the antagonist of the IGF-1R, or the antagonist of the IGF-1R and the immunoglobulin heavy chain constant region. Furthermore, the nucleic acid molecules optionally may also include a “leader” or “signal” sequence based upon, for example, an immunoglobulin light chain sequence fused directly to a hinge region of the immunoglobulin heavy chain constant region. The presence of the leader/signal sequences directs the chimeric polypeptide for secretion. The leader/signal sequence is cleaved upon secretion and is not present in the secreted chimeric polypeptide. In a particular embodiment, when the Fc region is based upon IgG sequences, the portion of the nucleic acid molecule encoding the Fc region includes, in a 5′ to 3′ direction, at least an immunoglobulin hinge region (i.e., a hinge region containing at least one cysteine amino acid capable of forming a disulfide bond with a second immunoglobulin hinge region sequence), an immunoglobulin CH2 domain and a CH3 domain. Furthermore, a nucleic acid molecule encoding the chimeric polypeptide may also be integrated within a replicable expression vector that may express the Fc fusion protein in, for example, a host cell.

In one embodiment, the immunoglobulin heavy chain constant region component of the chimeric polypeptide is non-immunogenic or is weakly immunogenic in the subject. The Fc region is considered non- or weakly immunogenic if the immunoglobulin heavy chain constant region fails to generate a detectable antibody response directed against the immunoglobulin heavy chain constant region. Accordingly, the immunoglobulin heavy chain constant region should be derived from immunoglobulins present, or based on amino acid sequences corresponding to immunoglobulins present in the same species as the intended recipient of the chimeric polypeptide.

The chimeric polypeptide of the present disclosure may be made using conventional methodologies known in the art. For example, the chimeric polypeptide constructs may be generated at the DNA level using recombinant DNA techniques, and the resulting nucleic acid molecule can be integrated into expression vectors, and expressed to produce the chimeric polypeptide. As used herein, the term “vector” is understood to mean any nucleic acid molecule comprising a nucleotide sequence competent to be incorporated into a host cell and to be recombined with and integrated into the host cell's chromosome, or to replicate autonomously as an episome. Such vectors include linear nucleic acid molecules, plasmids, phagemids, cosmids, RNA vectors, viral vectors and the like. Non-limiting examples of a viral vector include a retrovirus, an adenovirus a lentivirus and an adeno-associated virus. As used herein, the term “gene expression” or “expression” of the chimeric polypeptide, is understood to mean the transcription of a DNA sequence, translation of the mRNA transcript, and secretion of the chimeric protein product. As an alternative to the chimeric polypeptide by genetic engineering techniques, chemical conjugation using conventional chemical cross-linkers may be used to fuse the protein moieties of the chimeric moieties.

In the chimeric polypeptide of the present disclosure, it is contemplated that the carboxy-terminus of the first moiety is associated with the amino-terminus of the second carrier moiety. The association between the first and second moiety can be covalent and can be done by a peptide bound. In some embodiments, the association between the first and second moiety is direct. In alternative embodiments, the association between the first and second moiety may be indirect by including a linker moiety between these two moieties. The linker moiety can be an amino acid linker moiety. The linker moiety can be 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 amino acids or more. In an embodiment, the amino acid linker is a flexible linker. In still another embodiment, the amino acid linker can be a GS linker (e.g., and in some embodiments, having the following formula (GS)ₙGG in which n is any integer between 1 and 10). In a specific embodiment, the (GS)ₙGG linker has or comprises the amino acid of SEQ ID NO: 11 or 12, a variant thereof or a fragment thereof. In still a further embodiment, the amino acid linker has or comprises the amino acid sequence of SEQ ID NO: 10, a variant thereof or a fragment thereof. In still another embodiment, the chimeric polypeptide can be produced in a recombinant fashion by genetically engineering a recombinant host cell.

In an embodiment, the chimeric polypeptide comprises or consists of the amino acid sequence of SEQ ID NO: 4, a variant of the amino acid sequence of SEQ ID NO: 4 or a fragment of SEQ ID NO: 4. In still another embodiment, the chimeric polypeptide comprises a tetramer of the amino acid sequence of SEQ ID NO: 4, a variant of the amino acid sequence of SEQ ID NO: 4 or a fragment of SEQ ID NO: 4. In an embodiment, the chimeric polypeptide comprises or consists of the amino acid sequence of SEQ ID NO: 5, a variant of the amino acid sequence of SEQ ID NO: 5 or a fragment of SEQ ID NO: 5. In still another embodiment, the chimeric polypeptide comprises a tetramer of the amino acid sequence of SEQ ID NO: 5, a variant of the amino acid sequence of SEQ ID NO: 5 or a fragment of SEQ ID NO: 5. In an embodiment, the chimeric polypeptide comprises or consists of the amino acid sequence of SEQ ID NO: 6, a variant of the amino acid sequence of SEQ ID NO: 6 or a fragment of SEQ ID NO: 6. In still another embodiment, the chimeric polypeptide comprises a tetramer of the amino acid sequence of SEQ ID NO: 6, a variant of the amino acid sequence of SEQ ID NO: 6 or a fragment of SEQ ID NO: 6. In an embodiment, the chimeric polypeptide comprises or consists of the amino acid sequence of SEQ ID NO: 7, a variant of the amino acid sequence of SEQ ID NO: 7 or a fragment of SEQ ID NO: 7. In still another embodiment, the chimeric polypeptide comprises a tetramer of the amino acid sequence of SEQ ID NO: 7, a variant of the amino acid sequence of SEQ ID NO: 7 or a fragment of SEQ ID NO: 7. In an embodiment, the chimeric polypeptide comprises or consists of the amino acid sequence of SEQ ID NO: 8, a variant of the amino acid sequence of SEQ ID NO: 8 or a fragment of SEQ ID NO: 8. In still another embodiment, the chimeric polypeptide comprises a tetramer of the amino acid sequence of SEQ ID NO: 8, a variant of the amino acid sequence of SEQ ID NO: 8 or a fragment of SEQ ID NO: 8. In an embodiment, the chimeric polypeptide comprises or consists of the amino acid sequence of SEQ ID NO: 9, a variant of the amino acid sequence of SEQ ID NO: 9 or a fragment of SEQ ID NO: 9. In still another embodiment, the chimeric polypeptide comprises a tetramer of the amino acid sequence of SEQ ID NO: 9, a variant of the amino acid sequence of SEQ ID NO: 9 or a fragment of SEQ ID NO: 9.

The present disclosure also concerns variants and fragments of the antagonist of the IGF-1R described herein or of the chimeric polypeptide comprising same. Variants and fragment of the antagonist of the IGF-1R or of the chimeric polypeptide possess substantially the same biological activity but a different amino acid sequence then the wild-type antagonist of the IGF-1R or chimeric polypeptide. In one embodiment, the variant or fragment retains the ability to form α-α or β-β disulfide bridges. In some specific embodiments, the variant or fragment comprise α- and β-subunits and have the ability to multimerize to form a tetramer. In another embodiment, the variant or fragment which retains the disulfide bonds in the extracellular domain of the native (wild-type) IGF-1R and/or mimics the 3D conformation of the native (wild-type) IGF-1R. In a further embodiment, the variant or fragment retains binding specificity of the wild-type antagonist of the IGF-1R for IGF-I and/or IGF-II as compared to insulin. In a specific embodiment, the variant or fragment of the antagonist of the IGF-1R binds IGF-I and/or IGF-II with an affinity at least about 100-fold, 200-fold, 300-fold, 400-fold, 500-fold, 600-fold, 700-fold, 800-fold, 900-fold or at least about 1000-fold higher than its affinity for binding insulin.

A variant comprises at least one amino acid difference when compared to the amino acid sequence of the wild-type polypeptide. In an embodiment, the variant exhibits at least 50%, 55%, 60%, 65%, 70%, 75%, 80%, 85%, 90%, 95%, 96%, 97%, 98% or 99% of the biological activity of the wild-type antagonist of the IGF-1R or the wild-type chimeric protein. The antagonist of the IGF-1R “variants” have at least 50%, 55%, 60%, 65%, 70%, 75%, 80%, 85%, 90%, 95%, 96%, 97%, 98% or 99% sequence identity to the wild-type antagonist of the IGF-1R described herein or the chimeric polypeptide described herein. The term “percent identity”, as known in the art, is a relationship between two or more polypeptide sequences or two or more polynucleotide sequences, as determined by comparing the sequences. The level of identity can be determined conventionally using known computer programs. Identity can be readily calculated by known methods, including but not limited to those described in: Computational Molecular Biology (Lesk, A. M., ed.) Oxford University Press, NY (1988); Biocomputing: Informatics and Genome Projects (Smith, D. W., ed.) Academic Press, NY (1993); Computer Analysis of Sequence Data, Part I (Griffin, A. M., and Griffin, H. G., eds.) Humana Press, NJ (1994); Sequence Analysis in Molecular Biology (von Heinje, G., ed.) Academic Press (1987); and Sequence Analysis Primer (Gribskov, M. and Devereux, J., eds.) Stockton Press, NY (1991). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR Inc., Madison, Wis.). Multiple alignments of the sequences disclosed herein were performed using the Clustal method of alignment (Higgins and Sharp (1989) CABIOS. 5:151-153) with the default parameters (GAP PENALTY=10, GAP LENGTH PEN ALT Y=10). Default parameters for pairwise alignments using the Clustal method were KTUPLB 1, GAP PENALTY=3, WINDOW=5 and DIAGONALS SAVED=5.

The variant of the antagonist of the IGF-1R or of the chimeric polypeptide described herein may be (i) one in which one or more of the amino acid residues are substituted with a conserved or non-conserved amino acid residue (preferably a conserved amino acid residue) and such substituted amino acid residue may or may not be one encoded by the genetic code, or (ii) one in which one or more of the amino acid residues includes a substituent group, or (iii) one in which the mature polypeptide is fused with another compound, such as a compound to increase the half-life of the polypeptide (for example, polyethylene glycol), or (iv) one in which the additional amino acids are fused to the mature polypeptide for purification of the polypeptide.

A “variant” of the antagonist of the IGF-1R or of the chimeric polypeptide can be a conservative variant or an allelic variant. As used herein, a conservative variant refers to alterations in the amino acid sequence that do not adversely affect the biological functions of the polypeptide. A substitution, insertion or deletion is said to adversely affect the polypeptide when the altered sequence prevents or disrupts a biological function associated with the polypeptide. For example, the overall charge, structure or hydrophobic-hydrophilic properties of the polypeptide can be altered without adversely affecting its biological activity. Accordingly, the amino acid sequence can be altered, for example to render the polypeptide more hydrophobic or hydrophilic, without adversely affecting the biological activities of the polypeptide.

The variants disclosed herein can include one or more conservative amino acid substitutions or one or more non-conservative amino acid substitutions or both. Conservative substitutions typically include the substitution of one amino acid for another with similar characteristics, e.g., substitutions within the following groups: valine, glycine; glycine, alanine; valine, isoleucine, leucine; aspartic acid, glutamic acid; asparagine, glutamine; serine, threonine; lysine, arginine; and phenylalanine, tyrosine. Other conservative amino acid substitutions are known in the art and are included herein. Non-conservative substitutions, such as replacing a basic amino acid with a hydrophobic one, are also well-known in the art.

A fragment of the wild-type antagonist of the IGF-1R, the chimeric polypeptide or their variants exhibits the biological activity of the wild-type antagonist of the IGF-1R, the chimeric polypeptide or the variant (e.g., ability to bind and sequester the ligand(s) of the type I IGF-1R). In an embodiment, the fragment exhibits at least 50%, 55%, 60%, 65%, 70%, 75%, 80%, 85%, 90%, 95%, 96%, 97%, 98% or 99% of the biological activity of the wild-type antagonist of the IGF-1R, the chimeric polypeptide or the variant thereof. Polypeptide “fragments” have at least at least 500, 600, 700, 800, 900, 10000 or more consecutive amino acids of the wild-type antagonist of the IGF-1R, the chimeric polypeptide or the variant. A fragment comprises at least one less amino acid residue when compared to the amino acid sequence of the polypeptide and still possess the biological activity of the full-length polypeptide. In some embodiments, the “fragments” have at least 50%, 55%, 60%, 65%, 70%, 75%, 80%, 85%, 90%, 95%, 96%, 97%, 98% or 99% identity to the wild-type antagonist of the IGF-1R, the chimeric polypeptide or the variant thereof. In some embodiments, fragments of the polypeptides can be employed for producing the corresponding full-length polypeptide by peptide synthesis. Therefore, the fragments can be employed as intermediates for producing the full-length polypeptides.

The antagonist of the IGF-1R, variants thereof and fragments thereof can include additional modifications to increase their stability, such, for example, one or more non-peptide bonds (which replace the peptide bonds) in the polypeptide sequence. Also included are analogs that include residues other than naturally occurring L-amino acids, e.g., D-amino acids or non-naturally occurring or synthetic amino acids, e.g., β or γ amino acids.

### Immune Response Activating Agents

The antagonist of the IGF-1R can be used in combination with one or more immune response activating agent. In some embodiments, the antagonist of the IGF-1R can be used to potentiate the therapeutic effects of an immune response activating agent. As used in the context of the present disclosure, the expression “immune response activating agent” refers to a therapeutic agent or a combination of therapeutic agents capable of increasing the biological activity of immune response cells (lymphocytes such as, for example, B cells and T cells, macrophages, dendritic cells, natural killer cells, neutrophils, etc.) for stimulating the immune response against a cancer cell (which may be, in some embodiments, a metastatic cancer cell). In some embodiments, the increase in biological activity is observed in tumor-associated immune cells. This increase in biological activity does not have to be permanent, it can be transient.

In an embodiment, the immune response activating agent is an antibody or an antibody derivative which is specific for an immune cell (and in some embodiments, a polypeptide which is associated with a protein expressed on the surface of an immune cell or a ligand recognized by a protein expressed on the surface of an immune cell). Naturally occurring antibodies or immunoglobulins have a common core structure in which two identical light chains (about 24 kD) and two identical heavy chains (about 55 or 70 kD) form a tetramer. The amino-terminal portion of each chain is known as the variable (V) region and can be distinguished from the more conserved constant (C) regions of the remainder of each chain. Within the variable region of the light chain is a C-terminal portion known as the J region. Within the variable region of the heavy chain, there is a D region in addition to the J region. Most of the amino acid sequence variation in immunoglobulins is confined to three separate locations in the V regions known as hypervariable regions or complementarity determining regions (CDRs) which are directly involved in antigen binding. Proceeding from the amino-terminus, these regions are designated CDR1, CDR2 and CDR3, respectively. The CDRs are held in place by more conserved framework regions (FRs). Proceeding from the amino-terminus, these regions are designated FR1, FR2, FR3, and FR4, respectively. The locations of CDR and FR regions and a numbering system have been defined by Kabat et al. (Kabat, E. A. et al., Sequences of Proteins of Immunological Interest, Fifth Edition, U.S. Department of Health and Human Services, U.S. Government Printing Office (1991)).

The antibodies disclosed herein can be polyclonal or monoclonal antibodies. The antibodies disclosed herein also include antibody fragments. As used herein, a “fragment” of an antibody is a portion of an antibody that is capable of specifically recognizing the same epitope as the full version of the antibody. Antibody fragments include, but are not limited to, the antibody light chain, a single chain antibody, Fv, Fab, Fab′ and F(ab′)2 fragments. Such fragments can be produced by enzymatic cleavage or by recombinant techniques. For instance, papain or pepsin cleavage can be used to generate Fab or F(ab′)2 fragments, respectively. Antibodies can also be produced in a variety of truncated forms using antibody genes in which one or more stop codons have been introduced upstream of the natural stop site. For example, a chimeric gene encoding the heavy chain of an F(ab′)2 fragment can be designed to include DNA sequences encoding the CH1 domain and hinge region of the heavy chain.

The antibody disclosed herein can be an antibody derivative, such as, for example, a chimeric antibody and a humanized antibody. As used herein, the term “chimeric antibody” refers to an immunoglobulin comprising two regions from two distinct animals. As used herein, the term “humanized antibody” refers to a subsection of a “chimeric antibody” and includes an immunoglobulin that comprises both a region derived from a human antibody or immunoglobulin and a region derived from a non-human antibody or immunoglobulin. The action of humanizing an antibody consists in substituting a portion of a non-human antibody with a corresponding portion of a human antibody. For example, a humanized antibody as used herein could comprise a non-human region variable region (such as a region derived from a murine antibody) capable of specifically recognizing its target and a human constant region derived from a human antibody. In another example, the humanized immunoglobulin can comprise a heavy chain and a light chain, wherein the light chain comprises a complementarity determining region derived from an antibody of non-human origin which binds its target and a framework region derived from a light chain of human origin, and the heavy chain comprises a complementarity determining region derived from an antibody of non-human origin which binds its target and a framework region derived from a heavy chain of human origin. Antibody fragments can also be humanized. For example, a humanized light chain comprising a light chain CDR (i.e. one or more CDRs) of non-human origin and a human light chain framework region. In another example, a humanized immunoglobulin heavy chain can comprise a heavy chain CDR (i.e., one or more CDRs) of non-human origin and a human heavy chain framework region. The CDRs can be derived from a non-human immunoglobulin.

The antibodies or antibody derivatives of the present disclosure can be a monovalent antibody and, in some additional embodiments, can be a single-chain antibody.

The antibodies of the present disclosure can be provided in a chimeric protein form comprises an antibody moiety and a carrier protein moiety.

The antibody, antibody derivative and antibody fragment are specific for at least one antigen. As used in the context of the present disclosure, an antibody, an antibody derivative or an antibody fragment is specific for an antigen when it is able to discriminate specifically the antigen from other (related or unrelated antigens). In some embodiments, the antigen is present on various immune cells and as such, even though the antibody, its derivative or its target is specific for an antigen, they can bind with specificity to different immune cells.

In still further embodiments, the immune response activating agent can be an antagonistic antibody. The expression “antagonistic antibody” refers to an antibody, an antibody derivative or antibody fragment capable of reducing (e.g., decreasing, inhibiting or abrogating) the biological activity of a receptor present on an immune cell.

In some specific embodiments, the immune response activating agent can be antagonistic to a specific immune check-point. As used herein, the term “immune check-point” refers to a cell surface protein, present on immune cells, whose activity need to be reduced, abolished or inhibited to increase the biological activity of the immune cell to ultimately stimulate the immune response. In such embodiment, the immune response stimulating agent can be an antagonistic (monoclonal) antibody for the immune check-point or a (neutralizing) antibody to the ligand of the immune check-point. For example, the immune check-point can be the cytotoxic T-lymphocyte associated protein 4 (CTLA-4) protein and the immune response stimulating agent can be an anti-CTLA-4 antibody (such as ipilimumab or tremelimumab) or an anti-CTLA-4 ligand antibody. In another embodiment, the immune check-point can be the programmed cell death 1 (PD-1) protein and the immune response stimulating agent can be an anti-PD-1 antibody (such as, for example, pembrolizumab or nivolumab or pidilizumab), an anti-PD-1 ligand antibody (e.g., an anti-programmed death ligand 1 (PD-L1) antibody, such as, for example atezolizumab, avelumab or durvalumab), or anti-PD-2 ligand antibodies (e.g., an anti-programmed death ligand 2 (PD-L2) antibody). In still another example, the immune check-point can be the T-cell immunoglobulin and mucin-domain containing-3 (TIM-3) protein and the immune response stimulating agent can be an anti-TIM-3 antibody or an anti-TIM-3 ligand antibody. In yet another example, the immune check-point can be the lymphocyte-activation gene 3 (LAG-3) protein and the immune response stimulating agent can be an anti-LAG-3 antibody or an anti-LAG-3 ligand antibody. In another example, the immune check-point can be CD244 (also referred to as 2B4) and the immune response stimulating agent can be an anti-CD244 antibody or an anti-CD244 ligand antibody. In a further example, the immune check-point can be the T cell immunoreceptor with Ig and ITIM domains (TIGIT) protein and the immune response stimulating agent can be an anti-TIGIT antibody or an anti-TIGIT ligand antibody (such as, for example, an anti-CD155 antibody). In a further example, the immune check-point can be CD96 and the immune response stimulating agent can be an anti-CD96 antibody or an anti-CD96 ligand antibody (such as, for example, an anti-CD155 antibody). In still another example, the immune check-point can be V-domain Ig suppressor of T cell activation (VISTA) protein and the immune response stimulating agent can be an anti-VISTA antibody or an anti-VISTA ligand antibody. In yet another example, the immune check-point can be CD112R and the immune response stimulating agent can be an anti-CD112R antibody or an anti-CD112R ligand (such as, for example, an anti-CD112 antibody). In yet another example, the immune response stimulating agent can be an anti-IGF-IR antibody.

In another embodiment, the immune response stimulating agent can be an agonistic antibody. The expression “agonistic antibody” refers to an antibody capable of upregulating (e.g., increasing, potentiating or supplementing) the biological activity of a cell-surface receptor to ultimately upregulate the biological activity of the immune cell expressing such receptor and stimulate the immune response. In an example, the receptor can be a TNF receptor superfamily member 4 protein (referred to as TNFRSF4 or OX40) and the immune response stimulating agent can be an anti-TNFRSF4 antibody. In still another example, the receptor can be a TNF receptor superfamily member 9 protein (referred to as TNFRSF9 or CD137 or 41BB) and the immune response stimulating agent can be an anti-TNFRSF9 antibody. In still another example, the receptor can be a TNF receptor superfamily member 18 protein (referred to as TNFRSF18 or GITR) and the immune response stimulating agent can be an anti-TNFRSF18 antibody. In still another example, the receptor can be a CD27 protein and the immune response stimulating agent can be an anti-CD27 antibody. In still another example, the receptor can be a CD28 protein and the immune response stimulating agent can be an anti-CD28 antibody. In yet another example, the receptor can be a CD40 protein and the immune response stimulating agent can be an anti-CD40 antibody.

In another embodiment, the anti-cancer immune-stimulating agent can be an antagonistic antibody for a cell-surface protein (including a cell receptor) or a soluble protein (including a cell receptor ligand) expressed by tumor cells and/or immune cells whose activity need to be reduced, abolished or inhibited to increase the biological activity of immune cell against tumor cells. In an example, the cell surface protein can be a CD47 protein and the immune response stimulating agent can be an anti-CD47 antibody. In another example, the cell surface protein can be a macrophage colony-stimulating factor receptor (M-CSFR also known as CSF1R) and the immune response stimulating agent can be an anti-M-CSFR antibody. In still another example, the cell surface and soluble protein can be CD73 and the immune response stimulating agent can be an anti-CD73 antibody. In yet another example, the cell surface protein can be CD39 and the immune response stimulating agent can be an anti-CD39 antibody. In yet another example, the cell surface protein can be C—C chemokine receptor type 2 (CCR2 or CD192) and the immune response stimulating agent can be an anti-CCR2 antibody. In yet another example, the cell surface protein can be C—C chemokine receptor type 2 (CCR2 or CD192) and the immune response stimulating agent can be an anti-CCR2 antibody. In yet another example, the cell surface protein can be C—C chemokine receptor type 2 (CCR2 or CD192) and the immune response stimulating agent can be an anti-CCR2 antibody. In yet another example, the cell surface protein can be a mannose receptor (CD206) and the immune response stimulating agent can be an anti-CD206 antibody. In yet another example, the cell surface protein can be CD-163 and the immune response stimulating agent can be an anti-CD163 antibody. In still another example, the soluble protein can be CCL2 and the immune response stimulating agent can be an anti-CCL2 antibody. In still a further example, the soluble protein can be transforming growth factor β (TGFβ) and the immune response stimulating agent can be an anti-TGFβ antibody. In yet a further embodiment, the soluble protein can be interleukin-10 (IL-10) and the immune response stimulating agent can be an anti-IL-10 antibody. In still a further embodiment, the soluble protein can be interleukin-6 (IL-6) and the immune response stimulating agent can be an anti-IL-6 antibody. the soluble protein can be the vascular endothelial growth factor (VEGF) and the immune response stimulating agent can be an anti-VEGF antibody. In yet another example, the soluble protein can be a chemokine ligand 1 (CXCL1) and the immune response stimulating agent can be an anti-CXCL1 antibody. In yet another example, the soluble protein can be a chemokine ligand 2 (CXCL2) and the immune response stimulating agent can be an anti-CXCL2 antibody. In yet another example, the soluble protein can be arginase 1 (ARG1) and the immune response stimulating agent can be an anti-ARG1 antibody.

When the immune response stimulating agent is an antibody, it can be designed to be specific to one polypeptide (e.g., monospecific) or having a plural specificity to more than one polypeptide as described herewith. The immune response stimulating agent can be a single type of antibody for a single immune response stimulating target or a combination of more than one antibody each specific for the same or a different immune response stimulating target(s). For example, the immune response stimulating agent can include one, two, three, four, five or more different antibodies which can be specific to one, two, three, four, five or more different immune response stimulating targets.

In additional embodiments, the immune response stimulating agent can be a viral infection (e.g., oncolytic viral infection, for example, by talimogene laherparepvec; T-VEC)), an adoptive cellular therapy (e.g., an adoptive cell therapy with chimeric antigen receptor (CAR)-expressing T cells, an adoptive cell therapy with transgenic T cell receptor (TCR)-expressing T cells, an adoptive cell therapy with autologous tumor-infiltrating T cells and/or an adoptive cell therapy with allogeneic natural killer cells), a small molecule (e.g., adenosine receptor A2A antagonist, indoleamine 2, 3-dioxygenase (IDO) inhibitors, tryptophan-2,3-dioxygenase (TDO) inhibitors, arginase 1 inhibitors), a tumor vaccine (e.g., comprising tumor cells, antigen-presenting cells and/or mutated tumor antigenic peptides), an agonist to Toll-like receptors, an agonist to STING (stimulator of interferon genes), an anti-transforming growth factor-β antibody and/or a bi-specific antibody that redirect natural killer cell or T cell cytotoxicity to a defined tumor antigen or a combination of defined tumor antigens.

### Therapeutic Use of the Antagonist of the IGR-1R

The present disclosure concerns the use of the antagonist of the IGF-1R or the chimeric polypeptide comprising same to reduce the immune suppression or increase the immune toxicity in a tissue. Prior to its contact with the antagonist or the chimeric polypeptide comprising same, the tissue is in a state of immune suppression (e.g., the presence of immune suppressed cells) or immune tolerance (i.e. tolerance towards foreign antigens) because it has a reduced amount of (and in some embodiments it lacks) immune cells or antigen-presenting cells that can activate immune cells and/or it has a reduced amount of (and in some embodiments it lacks) immune cells which are capable of mounting a cytotoxic) immune response. In some embodiments, the tissue may be in a subject. In the methods of the present disclosure, it is not necessary that the subject exhibits an overall state of immune depression, but that, in one of its tissue, a state of immune suppression is observed. In additional embodiments, the tissue may include one or more microenvironment exhibiting a state of immune suppression or immune-tolerance or immune response blockade. As used in the context of the present disclosure, a “microenvironment” refers to a locus in a tissue which is smaller than the tissue itself and associated with a growing malignant entity. In some embodiment, the micro-environment can have a volume between 1 and 10 000 mm³. In still further embodiments, the tissue may include one or more malignant tumor which creates in its vicinity one or more microenvironment which exhibits a state of immune suppression. In some embodiments, the method comprises determining if the tissue (such as for example, the micro-environment in the vicinity of the malignant tumor) comprises a micro-environment exhibiting a state of immune suppression (or immune inactivity) or the subject to be treated is immunosuppressed prior to contacting it with the antagonist of the IGF-1R or the chimeric polypeptide.

The method comprises contacting the antagonist of the IGF-1R or the chimeric polypeptide with the tissue so as to reduce the immune suppression and increase the immune cytotoxicity in the contacted tissue. The reduction in the immune suppression and the increase in the immune cytotoxicity is observed with respect to a control tissue (which also exhibits a comparable state of immune suppression to the tissue prior to the contact) which was not contacted with the antagonist of the IGF-1R or the chimeric polypeptide. The antagonist of the IGF-1R or the chimeric polypeptide can be administered to a subject comprising the tissue so as to allow the contact between the tissue and the antagonist of the IGF-1R or the chimeric polypeptide. In some embodiments, the tissue is in vitro or ex vivo. In some embodiments, the tissue can be present in a subject. In further embodiments, the tissue can comprise one or more malignant tumor which creates, in its micro-environment, a state of immune suppression. In some embodiments, the malignant tumor is a metastatic tumor. In yet additional embodiments, the malignant tumor is from a pancreatic carcinoma. In yet additional embodiments, the malignant tumor is a liver metastasis (which can, in some embodiments, be from a pancreatic carcinoma).

In some embodiments, the method can be used to reduce the immune suppression by reducing the amount or the level of myeloid derived suppressor cells, immunosuppressive (N2) neutrophils and/or anti-inflammatory immunosuppressive (M2) tumor-associate macrophages in the tissue (for example in the micro-environment of the malignant tumor). As such, in some embodiments, the method can include determining in the tissue, prior and/or after the contact with the antagonist of the IGF-1R or the chimeric polypeptide, the amount or the level of immune suppressive cells such as, for example, myeloid derived suppressor cells, immunosuppressive (N2) neutrophils and/or anti-inflammatory immunosuppressive (M2) tumor-associate macrophages in the tissue While also increasing the antigen-presenting potential of dendritic cells. When the tissue is the liver and the immune suppression is caused, at least in part, by the presence of a metastasis (such as, for example, a liver metastasis of a pancreatic carcinoma), the method can be used to decrease the activation of hepatic stellate cells (or stromal cells) in the tissue. In additional embodiments, the methods can be used to decrease in the tissue CD11b+, Ly6G+ and Ly6C+ immune cells, CD163+ immune cells, and/or CD206+ immune cells (for example in the micro-environment of the malignant tumor). As such, in some embodiments, the method can include determining in the tissue, prior and/or after the contact with the antagonist of the IGF-1R or the chimeric polypeptide, the amount or the level of immune suppressive cells such as, for example, CD11b+, Ly6G+ and Ly6C+ immune cells, CD163+ immune cells and/or CD206+ immune cells in the tissue.

In some embodiments, the method can be used to increase the amount, the level or the activation of immune cells, such as for example, dendritic cells and/or increasing pro-inflammatory (N1) neutrophils in the tissue (for example in the micro-environment of the malignant tumor). As such, in some embodiments, the method can include determining in the tissue, prior and/or after the contact with the antagonist of the IGF-1R or the chimeric polypeptide, the amount or the level of immune response cells such as, for example, activated dendritic cells and/or increasing pro-inflammatory (N1) neutrophils in the tissue. In additional embodiments, the methods can be used to increase in the tissue the amount or the level of CD11b+, CD11c+ and MHCII+ immune-accessory cells, ICAM-1+ immune cells, CD4+ immune cells, CD8+ cells (including, but not limited to CD8+ and PD1+ cells) and/or CD68+ cells (for example in the micro-environment of the malignant tumor). As such, in some embodiments, the method can include determining in the tissue, prior and/or after the contact with the antagonist of the IGF-1R or the chimeric polypeptide, the amount or the level of immune cytotoxic cells such as, for example, CD11b+, CD11c+ and MHCII+ immune-accessory cells, ICAM-1+ immune cells, CD4+ immune cells, CD8+ cells (including, but not limited to CD8+ and PD1+ cells) and/or CD68+ cells in the tissue.

The methods of the present disclosure can be used to modulate the content of the tissue, and especially the micro-environment that exhibits the immune suppression so as to favor a state of immune activity and cytotoxicity with potential systemic effects. This modulation can be observed in, the cells present in the micro-environment and also in deposition of extracellular matrix proteins. In some embodiments, the method can be used to decrease the amount or the level of TGF-β, collagen I and/or α-smooth muscle actin. In other embodiments, the method can be used to increase the amount or the level of the IFN-γ and/or granzyme B. As such, in some embodiments, the method can include determining in the tissue, prior and/or after the contact with the antagonist of the IGF-1R or the chimeric polypeptide, the amount or the level of TGF-β, collagen I, α-smooth muscle actin, IFN-γ; and/or granzyme B in the tissue.

In the methods described herein, the antagonist of the IGF-1R or the chimeric polypeptide can be used alone or in combination with an immune response activating agent. In some embodiments, the antagonist of the IGF-1R or the chimeric polypeptide is intended to be contacted with a tissue or administered to a subject prior to, concomitantly or after having contacted/administered the immune response activating agent. In other embodiments, the immune response activating agent is intended to be contacted with a tissue or administered to a subject prior to, concomitantly or after having contacted/administered the antagonist of the IGF-1R or the chimeric polypeptide. The antagonist of the IGF-1R, the chimeric polypeptide or the immune response activating agent can be contacted with the tissue or administered to the subject in a pharmaceutically effective amount or therapeutically effective amount. These expressions refer to an amount (dose) effective in mediating a therapeutic benefit to a subject (for example reducing immune suppression, increasing immune cytotoxicity, treatment and/or alleviation of symptoms of cancer). It is also to be understood herein that a “pharmaceutically effective amount” may be interpreted as an amount giving a desired therapeutic effect, either taken in one dose or in any dosage or route, taken alone or in combination with other therapeutic agents.

The method can be used to prevent, alleviate the symptoms or treat conditions in which a tissue exhibits a state of immune suppression. Immunosuppression can be observed in various diseases or conditions such as cancers, endocrine disorders (such as acromegaly), diabetes, thyroid eye diseases, skin diseases (such as acne and psoriasis), auto-immune disorders (such as in cases of hyper immunity or multiple sclerosis) as well as frailty.

In some embodiments, the methods of the present disclosure the antagonist of the IGF-1R or the chimeric polypeptide (alone or in combination with the immune response activating agent) can be used in the prevention, treatment and alleviation of symptoms of a cancer. These expressions refer to the ability of a method, a therapeutic agent or a combination of therapeutic agents to limit the development, progression and/or symptomology of a cancer. Broadly, the prevention, treatment and/or alleviation of symptoms can encompass the reduction of proliferation of the cells (e.g., by reducing the total number of cells in an hyperproliferative state and/or by reducing the pace of proliferation of cells), the reduction of the immune suppression or the increase in the immune cytotoxicity. Symptoms associated with cancer, but are not limited to: local symptoms which are associated with the site of the primary cancer (such as lumps or swelling (tumor), hemorrhage, ulceration and pain), metastatic symptoms which are associated to the spread of cancer to other locations in the body (such as enlarged lymph nodes, hepatomegaly, splenomegaly, pain, fracture of affected bones, and neurological symptoms) and systemic symptoms (such as weight loss, fatigue, excessive sweating, anemia and paraneoplastic phenomena).

The cancer can be, for example, a pancreatic cancer (such as for example a pancreatic ductal adenocarcinoma). The cancer can be, for example, a glioma. The cancer can be, for example, a lung cancer (such as, for example, a non-small-cell lung cancer or a small-cell cancer), a breast cancer, a liver cancer (such as, for example, an hepatocellular carcinoma), a kidney/renal cancer (such as, for example, a renal cell carcinoma), a stomach cancer, a colorectal cancer, a head and neck tumor, an ovarian cancer, a bladder cancer, a skin cancer (such as, for example, a squamous cell carcinoma, a basal cell carcinoma, a Merkel cell carcinoma, a cutaneous melanoma or a uveal melanoma), an esophagus cancer, a fallopian tube cancer, a genitourinary tract cancer (such as, for example, a transitional cell carcinoma or an endometrioid carcinoma), a prostate cancer (such as, for example, a hormone refractory prostate cancer), a stomach cancer, a nasopharyngeal cancer (such as, for example, a nasopharyngeal carcinoma), a peritoneal cancer, an adrenal gland cancer, an anal cancer, a thyroid cancer (such as, for example, an anaplastic thyroid cancer), a biliary cancer (such as, for example, cholangiocarcinoma), a gastro-intestinal cancer, a mouth cancer, a nervous system cancer, a penis tumor and/or a thymic cancer. The cancer can be a melanoma, a sarcoma, a mesothelioma, a glioblastoma, a lymphoma (such as, for example, a B-cell lymphoma (including diffuse large B-cell lymphoma), Hodgkins disease, a non-Hodgkin lymphoma, a multiple myeloma, a follicle center lymphoma, a peripheral T-cell lymphoma, a primary mediastinal large B-cell lymphoma or a myelodysplastic syndrome), a leukemia (such as, for example, an acute myelogenous leukemia, a chronic lymphocytic leukemia or a chronic myelocytic leukemia), a glioma and/or a melanoma. The cancer can be a stage I cancer, a stage II cancer, a stage Ill cancer or a stage IV cancer. The cancer can be a metastatic cancer. The cancer can be a hormone-sensitive or a hormone-refractory cancer. In some embodiments, the method can include determining the presence of a cancer in the subject intended to receive the IGF-1R antagonist or the chimeric polypeptide (alone or in combination with the immune response activating agent) or having received at least one dose of the IGF-1R antagonist or the chimeric polypeptide (alone or in combination with the immune response activating agent). This determination step can be done to determine if additional doses of the IGF-1R antagonist or the chimeric polypeptide (alone or in combination with the immune response activating agent) should be administered to the subject.

TABLE 1
List of the biological sequences referred to in the present disclosure

| SEQ ID | | |
|---|---|---|
| NO: | Sequence | Description |
| 1 | MKSGSGGGSPTSLWGLLFLSAALSLWPTSGEICGPGIDIRN | Homo sapiens-Full |
| | DYQQLKRLENCTVIEGYLHILLISKAEDYRSYRFPKLTVITEYL | length IGF-1R |
| | LLFRVAGLESLGDLFPNLTVIRGWKLFYNYALVIFEMTNLKDI | |
| | GLYNLRNITRGAIRIEKNADLCYLSTVDWSLILDAVSNNYIVG | |
| | NKPPKECGDLCPGTMEEKPMCEKTTINNEYNYRCWTTNRC | |
| | QKMCPSTCGKRACTENNECCHPECLGSCSAPDNDTACVAC | |
| | RHYYYAGVCVPACPPNTYRFEGWRCVDRDFCANILSAESS | |
| | DSEGFVIHDGECMQECPSGFIRNGSQSMYCIPCEGPCPKVC | |
| | EEEKKTKTIDSVTSAQMLQGCTIFKGNLLINIRRGNNIASELE | |
| | NFMGLIEVVTGYVKIRHSHALVSLSFLKNLRLILGEEQLEGNY | |
| | SFYVLDNQNLQQLWDWDHRNLTIKAGKMYFAFNPKLCVSEI | |
| | YRMEEVTGTKGRQSKGDINTRNNGERASCESDVLHFTSTTT | |
| | SKNRIIITWHRYRPPDYRDLISFTVYYKEAPFKNVTEYDGQD | |
| | ACGSNSWNMVDVDLPPNKDVEPGILLHGLKPWTQYAVYVK | |
| | AVTLTMVENDHIRGAKSEILYIRTNASVPSIPLDVLSASNSSS | |
| | QLIVKWNPPSLPNGNLSYYIVRWQRQPQDGYLYRHNYCSK | |
| | DKIPIRKYADGTIDIEEVTENPKTEVCGGEKGPCCACPKTEA | |
| | EKQAEKEEAEYRKVFENFLHNSIFVPRPERKRRDVMQVANT | |
| | TMSSRSRNTTAADTYNITDPEELETEYPFFESRVDNKERTVI | |
| | SNLRPFTLYRIDIHSCNHEAEKLGCSASNFVFARTMPAEGAD | |
| | DIPGPVTWEPRPENSIFLKWPEPENPNGLILMYEIKYGSQVE | |
| | DQRECVSRQEYRKYGGAKLNRLNPGNYTARIQATSLSGNG | |
| | SWTDPVFFYVQAKTGYENFIHLIIALPVAVLLIVGGLVIMLYVF | |
| | HRKRNNSRLGNGVLYASVNPEYFSAADVYVPDEWEVAREKI | |
| | TMSRELGQGSFGMVYEGVAKGVVKDEPETRVAIKTVNEAA | |
| | SMRERIEFLNEASVMKEFNCHHVVRLLGVVSQGQPTLVIME | |
| | LMTRGDLKSYLRSLRPEMENNPVLAPPSLSKMIQMAGEIAD | |
| | GMAYLNANKFVHRDLAARNCMVAEDFTVKIGDFGMTRDIYE | |
| | TDYYRKGGKGLLPVRWMSPESLKDGVFTTYSDVWSFGVVL | |
| | WEIATLAEQPYQGLSNEQVLRFVMEGGLLDKPDNCPDMLFE | |
| | LMRMCWQYNPKMRPSFLEIISSIKEEMEPGFREVSFYYSEE | |
| | NKLPEPEELDLEPENMESVPLDPSASSSSLPLPDRHSGHKA | |
| | ENGPGPGVLVLRASFDERQPYAHMNGGRKNERALPLPQSS | |
| | TC | |
| 2 | MKSGSGGGSPTSLWGLLFLSAALSLWPTSGEICGPGIDIRN | Artificial sequence |
| | DYQQLKRLENCTVIEGYLHILLISKAEDYRSYRFPKLTVITEYL | SIGFIR933 |
| | LLFRVAGLESLGDLFPNLTVIRGWKLFYNYALVIFEMTNLKDI | |
| | GLYNLRNITRGAIRIEKNADLCYLSTVDWSLILDAVSNNYIVG | |
| | NKPPKECGDLCPGTMEEKPMCEKTTINNEYNYRCWTTNRC | |
| | QKMCPSTCGKRACTENNECCHPECLGSCSAPDNDTACVAC | |
| | RHYYYAGVCVPACPPNTYRFEGWRCVDRDFCANILSAESS | |
| | DSEGFVIHDGECMQECPSGFIRNGSQSMYCIPCEGPCPKVC | |
| | EEEKKTKTIDSVTSAQMLQGCTIFKGNLLINIRRGNNIASELE | |
| | NFMGLIEVVTGYVKIRHSHALVSLSFLKNLRLILGEEQLEGNY | |
| | SFYVLDNQNLQQLWDWDHRNLTIKAGKMYFAFNPKLCVSEI | |
| | YRMEEVTGTKGRQSKGDINTRNNGERASCESDVLHFTSTTT | |
| | SKNRIIITWHRYRPPDYRDLISFTVYYKEAPFKNVTEYDGQD | |
| | ACGSNSWNMVDVDLPPNKDVEPGILLHGLKPWTQYAVYVK | |
| | AVTLTMVENDHIRGAKSEILYIRTNASVPSIPLDVLSASNSSS | |
| | QLIVKWNPPSLPNGNLSYYIVRWQRQPQDGYLYRHNYCSK | |
| | DKIPIRKYADGTIDIEEVTENPKTEVCGGEKGPCCACPKTEA | |
| | EKQAEKEEAEYRKVFENFLHNSIFVPRPERKRRDVMQVANT | |
| | TMSSRSRNTTAADTYNITDPEELETEYPFFESRVDNKERTVI | |
| | SNLRPFTLYRIDIHSCNHEAEKLGCSASNFVFARTMPAEGAD | |
| | DIPGPVTWEPRPENSIFLKWPEPENPNGLILMYEIKYGSQVE | |
| | DQRECVSRQEYRKYGGAKLNRLNPGNYTARIQATSLSGNG | |
| | SWTDPVFFYVQAKTGYENF | |
| 3 | MKSGSGGGSPTSLWGLLFLSAALSLWPTSGEICGPGIDIRN | Artificial sequence |
| | DYQQLKRLENCTVIEGYLHILLISKAEDYRSYRFPKLTVITEYL | IGF-1R |
| | LLFRVAGLESLGDLFPNLTVIRGWKLFYNYALVIFEMTNLKDI | |
| | GLYNLRNITRGAIRIEKNADLCYLSTVDWSLILDAVSNNYIVG | |
| | NKPPKECGDLCPGTMEEKPMCEKTTINNEYNYRCWTTNRC | |
| | QKMCPSTCGKRACTENNECCHPECLGSCSAPDNDTACVAC | |
| | RHYYYAGVCVPACPPNTYRFEGWRCVDRDFCANILSAESS | |
| | DSEGFVIHDGECMQECPSGFIRNGSQSMYCIPCEGPCPKVC | |
| | EEEKKTKTIDSVTSAQMLQGCTIFKGNLLINIRRGNNIASELE | |
| | NFMGLIEVVTGYVKIRHSHALVSLSFLKNLRLILGEEQLEGNY | |
| | SFYVLDNQNLQQLWDWDHRNLTIKAGKMYFAFNPKLCVSEI | |
| | YRMEEVTGTKGRQSKGDINTRNNGERASCESDVLHFTSTTT | |
| | SKNRIIITWHRYRPPDYRDLISFTVYYKEAPFKNVTEYDGQD | |
| | ACGSNSWNMVDVDLPPNKDVEPGILLHGLKPWTQYAVYVK | |
| | AVTLTMVENDHIRGAKSEILYIRTNASVPSIPLDVLSASNSSS | |
| | QLIVKWNPPSLPNGNLSYYIVRWQRQPQDGYLYRHNYCSK | |
| | DKIPIRKYADGTIDIEEVTENPKTEVCGGEKGPCCACPKTEA | |
| | EKQAEKEEAEYRKVFENFLHNSIFVPRPERKRRDVMQVANT | |
| | TMSSRSRNTTAADTYNITDPEELETEYPFFESRVDNKERTVI | |
| | SNLRPFTLYRIDIHSCNHEAEKLGCSASNFVFARTMPAEGAD | |
| | DIPGPVTWEPRPENSIFLKWPEPENPNGLILMYEIKYGSQVE | |
| | DQRECVSRQEYRKYGGAKLNRLNPGNYTARIQATSLSGNG | |
| | SWTDPVFFYVQAKTGYENF | |
| 4 | MKSGSGGGSPTSLWGLLFLSAALSLWPTSGEICGPGIDIRN | Artificial sequence- |
| | DYQQLKRLENCTVIEGYLHILLISKAEDYRSYRFPKLTVITEYL | h-sIGF1R-Fc of U.S. |
| | LLFRVAGLESLGDLFPNLTVIRGWKLFYNYALVIFEMTNLKDI | Patent Ser. No. |
| | GLYNLRNITRGAIRIEKNADLCYLSTVDWSLILDAVSNNYIVG | 10538575 |
| | NKPPKECGDLCPGTMEEKPMCEKTTINNEYNYRCWTTNRC | |
| | QKMCPSTCGKRACTENNECCHPECLGSCSAPDNDTACVAC | |
| | RHYYYAGVCVPACPPNTYRFEGWRCVDRDFCANILSAESS | |
| | DSEGFVIHDGECMQECPSGFIRNGSQSMYCIPCEGPCPKVC | |
| | EEEKKTKTIDSVTSAQMLQGCTIFKGNLLINIRRGNNIASELE | |
| | NFMGLIEVVTGYVKIRHSHALVSLSFLKNLRLILGEEQLEGNY | |
| | SFYVLDNQNLQQLWDWDHRNLTIKAGKMYFAFNPKLCVSEI | |
| | YRMEEVTGTKGRQSKGDINTRNNGERASCESDVLHFTSTTT | |
| | SKNRIIITWHRYRPPDYRDLISFTVYYKEAPFKNVTEYDGQD | |
| | ACGSNSWNMVDVDLPPNKDVEPGILLHGLKPWTQYAVYVK | |
| | AVTLTMVENDHIRGAKSEILYIRTNASVPSIPLDVLSASNSSS | |
| | QLIVKWNPPSLPNGNLSYYIVRWQRQPQDGYLYRHNYCSK | |
| | DKIPIRKYADGTIDIEEVTENPKTEVCGGEKGPCCACPKTEA | |
| | EKQAEKEEAEYRKVFENFLHNSIFVPRPERKRRDVMQVANT | |
| | TMSSRSRNTTAADTYNITDPEELETEYPFFESRVDNKERTVI | |
| | SNLRPFTLYRIDIHSCNHEAEKLGCSASNFVFARTMPAEGAD | |
| | DIPGPVTWEPRPENSIFLKWPEPENPNGLILMYEIKYGSQVE | |
| | DQRECVSRQEYRKYGGAKLNRLNPGNYTARIQATSLSGNG | |
| | SWTDPVFFYVQAKTGYENFEFDISSTMVRSDKTHTCPPCPA | |
| | PELLGGPSVFLFPPKPKDTLMISRTPEVTCVVVDVSHEDPEV | |
| | KFNWYVDGVEVHNAKTKPREEQYNSTYRVVSVLTVLHQDW | |
| | LNGKEYKCKVSNKALPAPIEKTISKAKGQPREPQVYTLPPSR | |
| | EEMTKNQVSLTCLVKGFYPSDIAVEWESNGQPENNYKTTPP | |
| | VLDSDGSFFLYSKLTVDKSRWQQGNVFSCSVMHEALHNHY | |
| | TQKSLSLSPGK | |
| 5 | MKSGSGGGSPTSLWGLLFLSAALSLWPTSGEICGPGIDIRN | Artificial sequence- |
| | DYQQLKRLENCTVIEGYLHILLISKAEDYRSYRFPKLTVITEYL | SIGF1R-hFc-IgG2 of |
| | LLFRVAGLESLGDLFPNLTVIRGWKLFYNYALVIFEMTNLKDI | U.S. Patent Ser. |
| | GLYNLRNITRGAIRIEKNADLCYLSTVDWSLILDAVSNNYIVG | No. 10538575 |
| | NKPPKECGDLCPGTMEEKPMCEKTTINNEYNYRCWTTNRC | |
| | QKMCPSTCGKRACTENNECCHPECLGSCSAPDNDTACVAC | |
| | RHYYYAGVCVPACPPNTYRFEGWRCVDRDFCANILSAESS | |
| | DSEGFVIHDGECMQECPSGFIRNGSQSMYCIPCEGPCPKVC | |
| | EEEKKTKTIDSVTSAQMLQGCTIFKGNLLINIRRGNNIASELE | |
| | NFMGLIEVVTGYVKIRHSHALVSLSFLKNLRLILGEEQLEGNY | |
| | SFYVLDNQNLQQLWDWDHRNLTIKAGKMYFAFNPKLCVSEI | |
| | YRMEEVTGTKGRQSKGDINTRNNGERASCESDVLHFTSTTT | |
| | SKNRIIITWHRYRPPDYRDLISFTVYYKEAPFKNVTEYDGQD | |
| | ACGSNSWNMVDVDLPPNKDVEPGILLHGLKPWTQYAVYVK | |
| | AVTLTMVENDHIRGAKSEILYIRTNASVPSIPLDVLSASNSSS | |
| | QLIVKWNPPSLPNGNLSYYIVRWQRQPQDGYLYRHNYCSK | |
| | DKIPIRKYADGTIDIEEVTENPKTEVCGGEKGPCCACPKTEA | |
| | EKQAEKEEAEYRKVFENFLHNSIFVPRPERKRRDVMQVANT | |
| | TMSSRSRNTTAADTYNITDPEELETEYPFFESRVDNKERTVI | |
| | SNLRPFTLYRIDIHSCNHEAEKLGCSASNFVFARTMPAEGAD | |
| | DIPGPVTWEPRPENSIFLKWPEPENPNGLILMYEIKYGSQVE | |
| | DQRECVSRQEYRKYGGAKLNRLNPGNYTARIQATSLSGNG | |
| | SWTDPVFFYVQAKTGYENFERKCCVECPPCPAPPVAGPSV | |
| | FLFPPKPKDTLMISRTPEVTCVVVDVSHEDPEVQFNWYVDG | |
| | VEVHNAKTKPREEQFNSTFRVVSVLTVVHQDWLNGKEYKC | |
| | KVSNKGLPAPIEKTISKTKGQPREPQVYTLPPSREEMTKNQV | |
| | SLTCLVKGFYPSDIAVEWESNGQPENNYKTTPPMLDSDGSF | |
| | FLYSKLTVDKSRWQQGNVFSCSVMHEALHNHYTQKSLSLS | |
| | PGK | |
| 6 | MKSGSGGGSPTSLWGLLFLSAALSLWPTSGEICGPGIDIRN | Artificial sequence- |
| | DYQQLKRLENCTVIEGYLHILLISKAEDYRSYRFPKLTVITEYL | Chimeric polypeptide |
| | LLFRVAGLESLGDLFPNLTVIRGWKLFYNYALVIFEMTNLKDI | Mod#1 |
| | GLYNLRNITRGAIRIEKNADLCYLSTVDWSLILDAVSNNYIVG | Linker is underlined |
| | NKPPKECGDLCPGTMEEKPMCEKTTINNEYNYRCWTTNRC | Substituted serine |
| | QKMCPSTCGKRACTENNECCHPECLGSCSAPDNDTACVAC | residues are in bold |
| | RHYYYAGVCVPACPPNTYRFEGWRCVDRDFCANILSAESS | and identified with an |
| | DSEGFVIHDGECMQECPSGFIRNGSQSMYCIPCEGPCPKVC | asterix |
| | EEEKKTKTIDSVTSAQMLQGCTIFKGNLLINIRRGNNIASELE | |
| | NFMGLIEVVTGYVKIRHSHALVSLSFLKNLRLILGEEQLEGNY | |
| | SFYVLDNQNLQQLWDWDHRNLTIKAGKMYFAFNPKLCVSEI | |
| | YRMEEVTGTKGRQSKGDINTRNNGERASCESDVLHFTSTTT | |
| | SKNRIIITWHRYRPPDYRDLISFTVYYKEAPFKNVTEYDGQD | |
| | ACGSNSWNMVDVDLPPNKDVEPGILLHGLKPWTQYAVYVK | |
| | AVTLTMVENDHIRGAKSEILYIRTNASVPSIPLDVLSASNSSS | |
| | QLIVKWNPPSLPNGNLSYYIVRWQRQPQDGYLYRHNYCSK | |
| | DKIPIRKYADGTIDIEEVTENPKTEVCGGEKGPCCACPKTEA | |
| | EKQAEKEEAEYRKVFENFLHNSIFVPRPERKRRDVMQVANT | |
| | TMSSRSRNTTAADTYNITDPEELETEYPFFESRVDNKERTVI | |
| | SNLRPFTLYRIDIHSCNHEAEKLGCSASNFVFARTMPAEGAD | |
| | DIPGPVTWEPRPENSIFLKWPEPENPNGLILMYEIKYGSQVE | |
| | DQRECVSRQEYRKYGGAKLNRLNPGNYTARIQATSLSGNG | |
| | SWTDPVFFYVQAKTGYENFEFDISSTMVRSDKTHTS*PPS*P | |
| | APELLGGPSVFLFPPKPKDTLMISRTPEVTCVVVDVSHEDPE | |
| | VKFNWYVDGVEVHNAKTKPREEQYNSTYRVVSVLTVLHQD | |
| | WLNGKEYKCKVSNKALPAPIEKTISKAKGQPREPQVYTLPPS | |
| | REEMTKNQVSLTCLVKGFYPSDIAVEWESNGQPENNYKTTP | |
| | PVLDSDGSFFLYSKLTVDKSRWQQGNVFSCSVMHEALHNH | |
| | YTQKSLSLSPGK | |
| 7 | MKSGSGGGSPTSLWGLLFLSAALSLWPTSGEICGPGIDIRN | Artificial sequence- |
| | DYQQLKRLENCTVIEGYLHILLISKAEDYRSYRFPKLTVITEYL | Chimeric polypeptide |
| | LLFRVAGLESLGDLFPNLTVIRGWKLFYNYALVIFEMTNLKDI | Mod#2 |
| | GLYNLRNITRGAIRIEKNADLCYLSTVDWSLILDAVSNNYIVG | GS linker is |
| | NKPPKECGDLCPGTMEEKPMCEKTTINNEYNYRCWTTNRC | underlined |
| | QKMCPSTCGKRACTENNECCHPECLGSCSAPDNDTACVAC | |
| | RHYYYAGVCVPACPPNTYRFEGWRCVDRDFCANILSAESS | |
| | DSEGFVIHDGECMQECPSGFIRNGSQSMYCIPCEGPCPKVC | |
| | EEEKKTKTIDSVTSAQMLQGCTIFKGNLLINIRRGNNIASELE | |
| | NFMGLIEVVTGYVKIRHSHALVSLSFLKNLRLILGEEQLEGNY | |
| | SFYVLDNQNLQQLWDWDHRNLTIKAGKMYFAFNPKLCVSEI | |
| | YRMEEVTGTKGRQSKGDINTRNNGERASCESDVLHFTSTTT | |
| | SKNRIIITWHRYRPPDYRDLISFTVYYKEAPFKNVTEYDGQD | |
| | ACGSNSWNMVDVDLPPNKDVEPGILLHGLKPWTQYAVYVK | |
| | AVTLTMVENDHIRGAKSEILYIRTNASVPSIPLDVLSASNSSS | |
| | QLIVKWNPPSLPNGNLSYYIVRWQRQPQDGYLYRHNYCSK | |
| | DKIPIRKYADGTIDIEEVTENPKTEVCGGEKGPCCACPKTEA | |
| | EKQAEKEEAEYRKVFENFLHNSIFVPRPERKRRDVMQVANT | |
| | TMSSRSRNTTAADTYNITDPEELETEYPFFESRVDNKERTVI | |
| | SNLRPFTLYRIDIHSCNHEAEKLGCSASNFVFARTMPAEGAD | |
| | DIPGPVTWEPRPENSIFLKWPEPENPNGLILMYEIKYGSQVE | |
| | DQRECVSRQEYRKYGGAKLNRLNPGNYTARIQATSLSGNG | |
| | SWTDPVFFYVQAKTGYENFGGGGSGGGGSGGGGSGGGG | |
| | SGGDKTHTCPPCPAPELLGGPSVFLFPPKPKDTLMISRTPEV | |
| | TCVVVDVSHEDPEVKFNWYVDGVEVHNAKTKPREEQYNST | |
| | YRVVSVLTVLHQDWLNGKEYKCKVSNKALPAPIEKTISKAKG | |
| | QPREPQVYTLPPSREEMTKNQVSLTCLVKGFYPSDIAVEWE | |
| | SNGQPENNYKTTPPVLDSDGSFFLYSKLTVDKSRWQQGNV | |
| | FSCSVMHEALHNHYTQKSLSLSPGK | |
| 8 | MKSGSGGGSPTSLWGLLFLSAALSLWPTSGEICGPGIDIRN | Artificial sequence- |
| | DYQQLKRLENCTVIEGYLHILLISKAEDYRSYRFPKLTVITEYL | Chimeric polypeptide |
| | LLFRVAGLESLGDLFPNLTVIRGWKLFYNYALVIFEMTNLKDI | Mod#3 |
| | GLYNLRNITRGAIRIEKNADLCYLSTVDWSLILDAVSNNYIVG | GS linker is |
| | NKPPKECGDLCPGTMEEKPMCEKTTINNEYNYRCWTTNRC | underlined |
| | QKMCPSTCGKRACTENNECCHPECLGSCSAPDNDTACVAC | Substituted serine |
| | RHYYYAGVCVPACPPNTYRFEGWRCVDRDFCANILSAESS | residues are in bold |
| | DSEGFVIHDGECMQECPSGFIRNGSQSMYCIPCEGPCPKVC | and identified with an |
| | EEEKKTKTIDSVTSAQMLQGCTIFKGNLLINIRRGNNIASELE | asterix |
| | NFMGLIEVVTGYVKIRHSHALVSLSFLKNLRLILGEEQLEGNY | |
| | SFYVLDNQNLQQLWDWDHRNLTIKAGKMYFAFNPKLCVSEI | |
| | YRMEEVTGTKGRQSKGDINTRNNGERASCESDVLHFTSTTT | |
| | SKNRIIITWHRYRPPDYRDLISFTVYYKEAPFKNVTEYDGQD | |
| | ACGSNSWNMVDVDLPPNKDVEPGILLHGLKPWTQYAVYVK | |
| | AVTLTMVENDHIRGAKSEILYIRTNASVPSIPLDVLSASNSSS | |
| | QLIVKWNPPSLPNGNLSYYIVRWQRQPQDGYLYRHNYCSK | |
| | DKIPIRKYADGTIDIEEVTENPKTEVCGGEKGPCCACPKTEA | |
| | EKQAEKEEAEYRKVFENFLHNSIFVPRPERKRRDVMQVANT | |
| | TMSSRSRNTTAADTYNITDPEELETEYPFFESRVDNKERTVI | |
| | SNLRPFTLYRIDIHSCNHEAEKLGCSASNFVFARTMPAEGAD | |
| | DIPGPVTWEPRPENSIFLKWPEPENPNGLILMYEIKYGSQVE | |
| | DQRECVSRQEYRKYGGAKLNRLNPGNYTARIQATSLSGNG | |
| | SWTDPVFFYVQAKTGYENFGGGGSGGGGSGGGGSGGGG | |
| | SGGDKTHTS*PPS*PAPELLGGPSVFLFPPKPKDTLMISRTPE | |
| | VTCVVVDVSHEDPEVKFNWYVDGVEVHNAKTKPREEQYNS | |
| | TYRVVSVLTVLHQDWLNGKEYKCKVSNKALPAPIEKTISKAK | |
| | GQPREPQVYTLPPSREEMTKNQVSLTCLVKGFYPSDIAVEW | |
| | ESNGQPENNYKTTPPVLDSDGSFFLYSKLTVDKSRWQQGN | |
| | VFSCSVMHEALHNHYTQKSLSLSPGK | |
| 9 | MKSGSGGGSPTSLWGLLFLSAALSLWPTSGEICGPGIDIRN | Artificial sequence- |
| | DYQQLKRLENCTVIEGYLHILLISKAEDYRSYRFPKLTVITEYL | Chimeric polypeptide |
| | LLFRVAGLESLGDLFPNLTVIRGWKLFYNYALVIFEMTNLKDI | Mod#4 |
| | GLYNLRNITRGAIRIEKNADLCYLSTVDWSLILDAVSNNYIVG | GS linker is |
| | NKPPKECGDLCPGTMEEKPMCEKTTINNEYNYRCWTTNRC | underlined |
| | QKMCPSTCGKRACTENNECCHPECLGSCSAPDNDTACVAC | |
| | RHYYYAGVCVPACPPNTYRFEGWRCVDRDFCANILSAESS | |
| | DSEGFVIHDGECMQECPSGFIRNGSQSMYCIPCEGPCPKVC | |
| | EEEKKTKTIDSVTSAQMLQGCTIFKGNLLINIRRGNNIASELE | |
| | NFMGLIEVVTGYVKIRHSHALVSLSFLKNLRLILGEEQLEGNY | |
| | SFYVLDNQNLQQLWDWDHRNLTIKAGKMYFAFNPKLCVSEI | |
| | YRMEEVTGTKGRQSKGDINTRNNGERASCESDVLHFTSTTT | |
| | SKNRIIITWHRYRPPDYRDLISFTVYYKEAPFKNVTEYDGQD | |
| | ACGSNSWNMVDVDLPPNKDVEPGILLHGLKPWTQYAVYVK | |
| | AVTLTMVENDHIRGAKSEILYIRTNASVPSIPLDVLSASNSSS | |
| | QLIVKWNPPSLPNGNLSYYIVRWQRQPQDGYLYRHNYCSK | |
| | DKIPIRKYADGTIDIEEVTENPKTEVCGGEKGPCCACPKTEA | |
| | EKQAEKEEAEYRKVFENFLHNSIFVPRPERKRRDVMQVANT | |
| | TMSSRSRNTTAADTYNITDPEELETEYPFFESRVDNKERTVI | |
| | SNLRPFTLYRIDIHSCNHEAEKLGCSASNFVFARTMPAEGAD | |
| | DIPGPVTWEPRPENSIFLKWPEPENPNGLILMYEIKYGSQVE | |
| | DQRECVSRQEYRKYGGAKLNRLNPGNYTARIQATSLSGNG | |
| | SWTDPVFFYVQAKTGYENFGGGGSGGGGSGGGGSGGGG | |
| | SGGGGSGG | |
| | CPAPELLGGPSVFLFPPKPKDTLMISRTPEVTCVVVDVSHED | |
| | PEVKFNWYVDGVEVHNAKTKPREEQYNSTYRVVSVLTVLH | |
| | QDWLNGKEYKCKVSNKALPAPIEKTISKAKGQPREPQVYTL | |
| | PPSREEMTKNQVSLTCLVKGFYPSDIAVEWESNGQPENNY | |
| | KTTPPVLDSDGSFFLYSKLTVDKSRWQQGNVFSCSVMHEA | |
| | LHNHYTQKSLSLSPGK | |
| 10 | EFDISSTMVRS | Artificial sequence- |
| | | linker present in the |
| | | amino acid sequence |
| | | of SEQ ID NO: 6 |
| 11 | GGGGSGGGGSGGGGSGGGGSGG | Artificial sequence- |
| | | linker present in the |
| | | amino acid sequence |
| | | of SEQ ID NO: 7 and |
| | | 8 |
| 12 | GGGGSGGGGSGGGGSGGGGSGGGGSGG | Artificial sequence- |
| | | linker present in the |
| | | amino acid sequence |
| | | of SEQ ID NO: 9 |
| 13 | DKTHTSPPSPAPELLGGPSVFLFPPKPKDTLMISRTPEVTCV | Artificial sequence- |
| | VVDVSHEDPEVKFNWYVDGVEVHNAKTKPREEQYNSTYRV | Fc portion of SEQ ID |
| | VSVLTVLHQDWLNGKEYKCKVSNKALPAPIEKTISKAKGQPR | NO : 6 |
| | EPQVYTLPPSREEMTKNQVSLTCLVKGFYPSDIAVEWESNG | |
| | QPENNYKTTPPVLDSDGSFFLYSKLTVDKSRWQQGNVFSC | |
| | SVMHEALHNHYTQKSLSLSPGK | |
| 14 | DKTHTCPPCPAPELLGGPSVFLFPPKPKDTLMISRTPEVTCV | Artificial sequence- |
| | VVDVSHEDPEVKFNWYVDGVEVHNAKTKPREEQYNSTYRV | Fc portion of SEQ ID |
| | VSVLTVLHQDWLNGKEYKCKVSNKALPAPIEKTISKAKGQPR | NO: 7 |
| | EPQVYTLPPSREEMTKNQVSLTCLVKGFYPSDIAVEWESNG | |
| | QPENNYKTTPPVLDSDGSFFLYSKLTVDKSRWQQGNVFSC | |
| | SVMHEALHNHYTQKSLSLSPGK | |
| 15 | DKTHTSPPSPAPELLGGPSVFLFPPKPKDTLMISRTPEVTCV | Artificial sequence- |
| | VVDVSHEDPEVKFNWYVDGVEVHNAKTKPREEQYNSTYRV | Fc portion of SEQ ID |
| | VSVLTVLHQDWLNGKEYKCKVSNKALPAPIEKTISKAKGQPR | NO: 8 |
| | EPQVYTLPPSREEMTKNQVSLTCLVKGFYPSDIAVEWESNG | |
| | QPENNYKTTPPVLDSDGSFFLYSKLTVDKSRWQQGNVFSC | |
| | SVMHEALHNHYTQKSLSLSPGK | |
| 16 | CPAPELLGGPSVFLFPPKPKDTLMISRTPEVTCVVVDVSHED | Artificial sequence- |
| | PEVKFNWYVDGVEVHNAKTKPREEQYNSTYRVVSVLTVLH | Fc portion of SEQ ID |
| | QDWLNGKEYKCKVSNKALPAPIEKTISKAKGQPREPQVYTL | NO: 9 |
| | PPSREEMTKNQVSLTCLVKGFYPSDIAVEWESNGQPENNY | |
| | KTTPPVLDSDGSFFLYSKLTVDKSRWQQGNVFSCSVMHEA | |
| | LHNHYTQKSLSLSPGK | |

The antagonist of the IGF-1R or the chimeric polypeptide (alone or in combination with the immune response activating agent) can be used/administered in various subjects, including, but not limited to, mammals such as humans.

### Example

Cells. The pancreatic ductal adenocarcinoma (PDAC) LMP cell line originated from a tumor that arose in the genetically engineered Kras G12D/+; p53R172H/+; Pdx1 Cre (KPC) mouse model, as described in detail elsewhere (Tseng et al., 2010). In syngeneic B6.129 F1 mice implanted in the pancreas with LMP cells, tumor growth and metastasis mimic the aggressive clinical behavior of PDAC. Murine pancreatic cancer cells KPC FC1199, referred to as FC1199, were generated in the Tuveson laboratory (Cold Spring Harbor Laboratory, New York, USA) from PDA tumor tissues obtained from KPC mice of a pure C57BL/6 background, as described previously (Hingorani et al., 2005), and were a generous gift from the Tuveson laboratory. All cell lines were routinely tested for common murine pathogens and mycoplasma contamination, as per the McGill University Animal Care Committee and the McGill University Biohazard Committee guidelines. The cells were routinely grown in a humidified incubator at 37° C. with 5% CO₂ in DMEM (Thermo Fischer scientific, Burlington, Canada) supplemented with 100 U/mL penicillin and 100 μg/mL streptomycin solution (Sigma), and 10% fetal bovine serum (Thermo Fischer scientific, Canada).

Animals. All mouse experiments were carried out in strict accordance with the guidelines of the Canadian Council on Animal Care (CCAC) “Guide to the Care and Use of Experimental Animals” and under the conditions and procedures approved by the Animal Care Committee of McGill University (AUP number: 5260). Mouse experiments were performed in male and female B6.129 F1 mice that are syngeneic to the LMP cells or in C57BI/6 male mice that are syngeneic to the FC1199 cells. BL6-Col-GFP mice in which type I collagen is genetically tagged with GFP were a kind gift from Dr. Tatiana Kisseleva (Department of Surgery, University of California, San Diego, La Jolla, CA, USA). They were backcrossed for one generation onto the 129S1/Svlmj (Jackson laboratories) background to obtain heterozygous Col-GFP mice that were crossbred with BL6 mice to generate first generation B16.129-Col-GFP F1 mice used for the analysis of activated HSC. All mice were bred in the animal facility of the Research Institute of the McGill University Health Center and used for the experiments at the ages of 7-12 weeks old.

Spontaneous PDAC liver metastasis. Spontaneous PDAC liver metastases were observed following the intra-pancreatic implantation of 1×10⁶ LMP cells in 25 μl Matrigel (Corning, NY, USA) mixed with 25 μl PBS, as previously described (Jiang et al., 2014). Animals were euthanized 21 days post tumor implantation, at which time metastases were visible on the surface of the liver and were enumerated and sized without prior fixation.

Experimental liver metastasis. Experimental liver metastases were generated by intrasplenic/portal injections of 1×10⁵ or 5×10⁵ tumor cells (as indicated), followed by splenectomy as previously described (Ham et al., 2015). Animals were euthanized 21 days later, and visible metastases on the surface of the liver were enumerated and sized without prior fixation. Where indicated, fragments of the livers were also fixed in 10% phosphate buffered formalin, paraffin embedded, and 5 μm sections stained with hematoxylin and eosin to detect micro-metastases and quantify the metastatic burden, as shown.

Immunostaining and confocal microscopy. B6129F1 mice were injected via the intrasplenic/portal route with 1×10⁵ or 5×10⁵ LMP cells as indicated, and the livers perfused at the time intervals indicated, first with PBS and then with 4 ml of a 4% paraformaldehyde solution. The perfused livers were placed in 4% paraformaldehyde for 48 h and then in 30% sucrose for an additional 48 h before they were stored at −80° C. For immunostaining, 10 μm cryostat sections were prepared, incubated first in a blocking solution (1% BSA and 1% FBS in PBS) and then for 1 h each with the primary antibodies, used at the indicated dilutions, and the appropriate Alexa Fluor-conjugated secondary antibodies The antibodies used in this study are listed in Table 2, all at room temperature (RT). Sections stained with the secondary antibodies only were used as controls in all the experiments. After washing with PBS, an autofluorescence quenching kit (Vector®TrueVIEW™, Burlingame, CA, USA) was used to reduce tissue autofluorescence and sections counterstained with 1 mg/ml DAPI (4,6-Diamidino-2-Phenylindole, dihydrochloride, Invitrogen, Eugene, OR, USA). The sections were mounted in the Prolong Gold anti-fade reagent (Molecular Probes, Eugene, Oregon, USA) and confocal images were captured with a Zeiss LSM-880 microscope with a spectrum detection capability. The immunostained cells were quantified blindly in at least 8 images acquired per section, per group.

TABLE 2
Primers and Antibodies used in this Example
Flow Cytometry Antibodies

| | | Catalog | |
|---|---|---|---|
| Name | Company | number | Dilution |
| CD45 BV510 | Biolegend | 103138 | 1/100 |
| CD11b PE | BD Pharmigen | 557397 | 1/1000 |
| CD11c PE-Cy5.5 | eBioscience | 35-0114-82 | 1/100 |
| Ly6C PerCP-Cy5.5 | eBioscience | 45-5932-80 | 1/1000 |
| Ly6G PE-Cy7 | eBioscience | 25-5931-82 | 1/1000 |
| ICAM1 APC | Invitrogen | A15396 | 1/200 |
| CD3e APC | BD Pharmigen | 553066 | 1/100 |
| CD4 FITC | eBioscience | 11-0041-82 | 1/100 |
| CD8a BV650 | BD Horizon | 563234 | 1/100 |
| MHC II (I-A/I-E) AF700 | eBioscience | 56-5321-82 | 1/100 |
| IFN-y PE | BD Pharmigen | 554412 | 1/75 |
| PD-1 (CD279) PE/Cy7 | Biolegend | 109110 | 1/100 |
| CTLA4 (CD152) PE | BD Pharmigen | 553720 | 1/100 |
| Lag3 (CD223) APC- | eBioscience | 47-2231-82 | 1/100 |
| eFluor780 | | | |

qPCR Primer Sequences

| | | |
|---|---|---|
| Gene name | Sequence Forward | Sequence Reverse |

| | | |
|---|---|---|
| Arg1 | GGAAGACAGCAGAGGAGGTG | TATGGTTACCCTCCCGTTGA |
| | (SEQ ID NO: 17) | (SEQ ID NO: 18) |
| VEGF | TTGAGACCCTGGTGGACATC | CACACAGGACGGCTTGAAGA |
| | (SEQ ID NO: 19) | (SEQ ID NO: 20) |
| GrzB | CCTCCTGCTACTGCTGAC | GTCAGCACAAAGTCCTCTC |
| | (SEQ ID NO: 21) | (SEQ ID NO: 22) |
| GAPDH | ACCCAGAAGACTGTGGATGG | ACCCAGAAGACTGTGGATGG |
| | (SEQ ID NO: 23) | (SEQ ID NO: 24) |

IHC Antibodies

| | | Catalog | |
|---|---|---|---|
| Name | Company | number | Dilution |
| CD4 | Biolegend | 100402 | 1/100 |
| CD8 | eBioscience | 550281 | 1/100 |
| F4/80 | CEDARLANE | CL8940AP | 1/100 |
| CD206 | Invitrogen | PA5-46994 | 1/100 |
| CD11c | Biolegend | 117312 | 1/100 |
| MHCII | abcam | ab25333 | 1/100 |
| Ki67 | abcam | ab16667 | 1/100 |
| aSMA | Dako | M0851 | 1/100 |
| PD-1 | R&D SYSTEMS | AF1021 | 1/100 |
| DAPI | Invitrogen | D1306 | 1/2000 |

Isolation of hepatic immune cells and flow cytometry. To analyze early changes in the TIME, mice were injected with 1×10⁵ tumor cells via the intrasplenic/portal route and the livers removed 14 days later (or as indicated). Liver homogenates were prepared in cold PBS and filtered through a stainless steel mesh, using a plunger. The filtrates were centrifuged at 500 rpm to separate the hepatocytes, the supernatants containing the non-parenchymal cell fraction centrifuged at 1400 rpm and the pellets resuspended in 10 ml of a 37.5% Percoll solution in HBSS containing 100 U/ml heparin and centrifuged at 1910 rpm for 30 minutes to obtain the immune cell-rich fraction. Prior to flow cytometry, red blood cells were removed using the ACK (ammonium chloride-potassium) solution and 1×10⁶ cells were immunostained with the indicated antibodies. Data acquisition was with a BD LSRFortessa and FACS Diva software and the data analyzed using the FlowJo software. For FC on hepatic leukocytes, single cells were gated based on size (FSC), granularity (SSC), viability using an eFluor™ 780 fixable dye (eBioscience™, Thermofisher) and the expression of CD45.

Ex vivo T cell activation for analysis of INF-γ production. Experimental liver metastases were generated by injecting 1×10⁵ LMP cells via the intrasplenic/portal route. Livers were resected 14 days later and immune cell isolated and stimulated for 4 h with phorbol-12-myristate-13-acetate (PMA; 5 ng/ml; Sigma) and ionomycin (500 ng/ml; Sigma) in the presence of a protein transport inhibitor (BD GolgiStop™). The activated T cells were first immunostained for extracellular markers and then fixed and permeabilized for IFN-γ staining prior to analysis by FC.

RNA extraction and qPCR. RNA was extracted from G-MDSC cells and CD3+CD8+ T cells using TRIzol (Ambion, Life Technologies). cDNA was synthesized from isolated RNA using a High Capacity cDNA Reverse Transcription Kit (Applied Biosystems, CA, USA), as per the manufacturer's protocol. qPCR was performed in a Bio-Rad Light Cycler (Bio-Rad, Hercules, CA, USA), using SYBR (Roche, ON, Canada). Two μg of total RNA were reverse transcribed and the cDNA analyzed using the primer sets listed in Table 2. Changes in expression levels were calculated using the ΔΔCt values and GAPDH was used to normalize for loading.

T cell suppression assay. Splenocytes from naive mice were isolated and red blood cells lysed as described above. Splenic CD3+ T cells were sorted by FACS, stained with CellTrace™ CFSE (ThermoFisher Scientific) and incubated for 48 h in RPMI with Dynabeads® Mouse T-Activator CD3/CD28 (ThermoFisher Scientific) in a 96-well plate at 37° C. Liver-derived MDSC from mice which were pre-treated, or not, with the IGF-Trap were isolated 14 days post-tumor injection and sorted as described above. MDSC were then added to the pre-activated splenic T cells at a ratio of 1:1. In the control condition, no MDSC were added to the activated T cells. After 48 h of co-incubation, the cells were harvested and analysis of CFSE intensity was performed using the BD LSRFortessa.

Data presentation and statistical analyses. The non-parametric Mann-Whitney test was used to analyze all metastasis data and a 2-tailed Student t-test was used to analyze ex vivo and in vitro data and the IF results. Box-and whiskers plots were used to show individual values, where applicable. Where indicated, the middle bar denotes the median value, the box limits extend from the 25^{th} to 75^{th} percentiles and the whiskers denote the lowest and highest values.

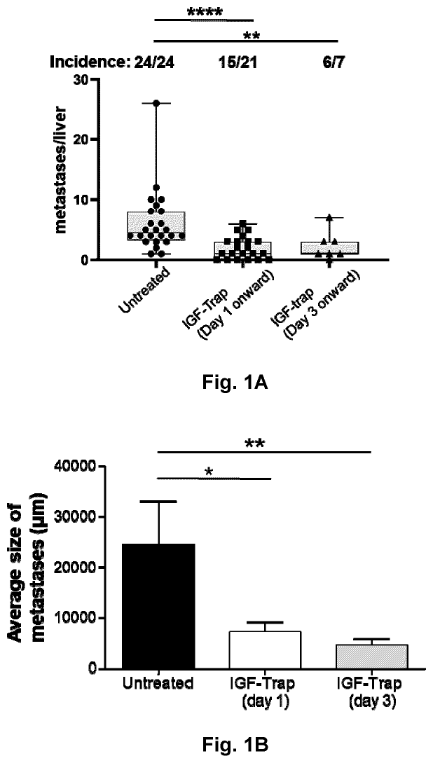

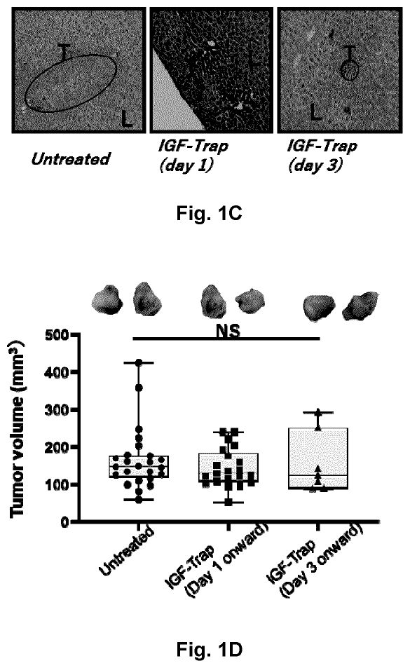

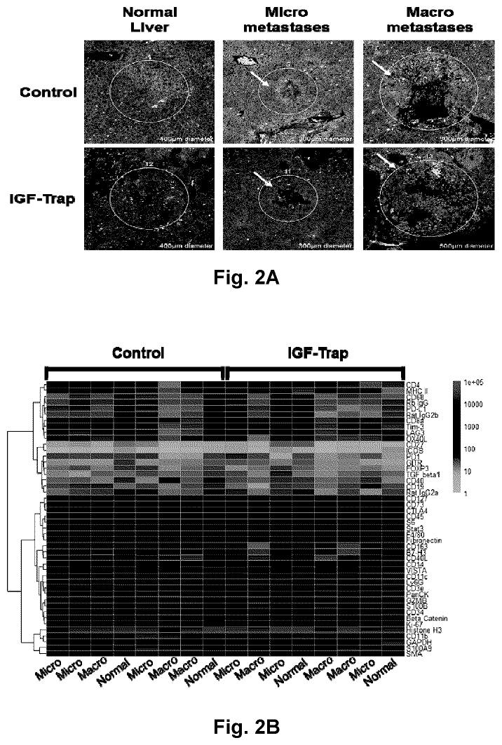

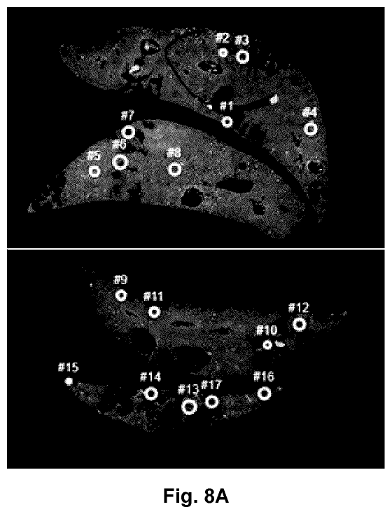

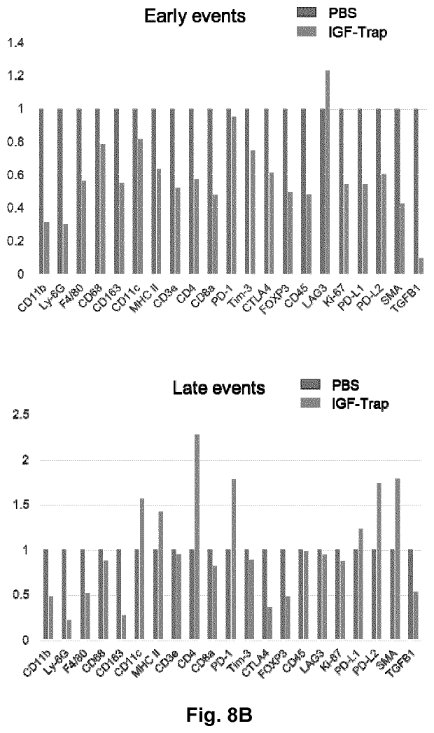

The IGF-Trap alters the microenvironment of PDAC liver metastases. It was previously shown that treatment with the IGF-Trap altered the TME in the liver (Rayes et al., 2018; Fernandez et al., 2017) and significantly reduced the outgrowth of liver metastases in several pre-clinical models of aggressive carcinomas (Wang et al., 2015; Vaniotis et al., 2018). The murine LMP cells originated from the KPC model of spontaneous pancreatic cancer (Frese et al., 2007). When implanted orthotopically into the pancreas of syngeneic BL6129 F1 mice, these cells mimic the pathology of the human disease and metastasize spontaneously to the liver, eventually causing accumulation of ascites and morbidity within 3-4 weeks following implantation (Milette et al., 2019; Tseng et al., 2010). The effect of the IGF-Trap on the growth of LMP cells in vivo and found that while this treatment did not alter local tumor growth in the pancreas (FIG. 1A), it had a marked effect on liver metastases, significantly reducing their numbers and sizes (FIG. 1B-D). This suggested that in this model, the IGF-Trap may have exerted its effect on hepatic metastases by altering the TME in the liver. To further investigate this possibility, the immune microenvironment in the livers of LMP-injected and IGF-Trap treated (or non-treated) mice was profiled using the NanoString GeoMx digital spatial profiler, in order to identify global changes in the expression of immune cell surface markers in the TIME that resulted from IGF-Trap treatment. The TIME associated with both micro-metastatic lesions and large diffuse liver metastases was analyzed in order to distinguish early and late events in immune cell recruitment, and within each liver section, compared the intensity of each signal to that associated with “tumor-free” areas within the same sections, in order to identify changes specific to tumor-infiltrating immune cells. When relative signal intensities in IGF-Trap and phosphate-buffered saline (PBS)-treated controls were compared, significant differences were observed in the accumulation of several immune cell subtypes in the TME of IGF-Trap treated, as compared to control mice (FIGS. 2A and B). Notable among them were a significant increase in the expression of CD11c and MHCII in both small and large metastases, consistent with increased recruitment and activation of DC, decreased accumulation of CD11b+ and Ly6G+ cells, consistent with a reduction in bone marrow derived granulocytic cells (neutrophils or granulocytic (G)-MDSC), increased presence of CD4+ T cell that was accompanied by increased expression of several immune checkpoints (PD-1, TiM3 and LAG-3) in early stage metastases, and an increase in CD68+ cells accompanied by reduced CD163 expression in the larger metastases, indicative of a reduction in M2 tumor-associated macrophages. In addition, this analysis also revealed a marked reduction in TGF-β1 levels in and around the metastases, consistent with an overall reduction in immunosuppressive signals (see FIG. 2B and FIG. 8B).

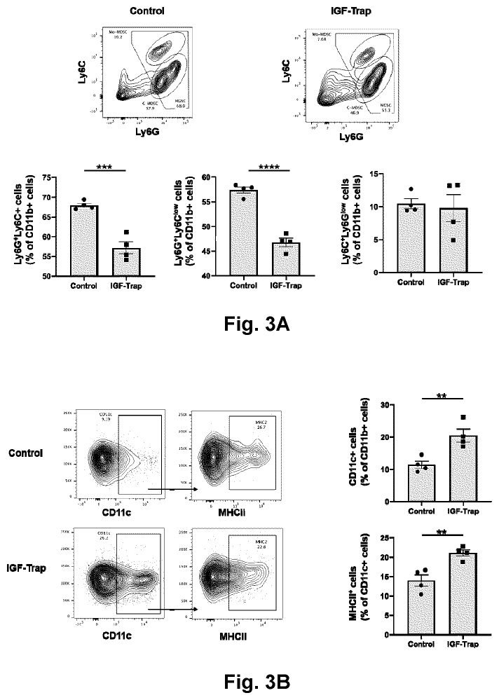

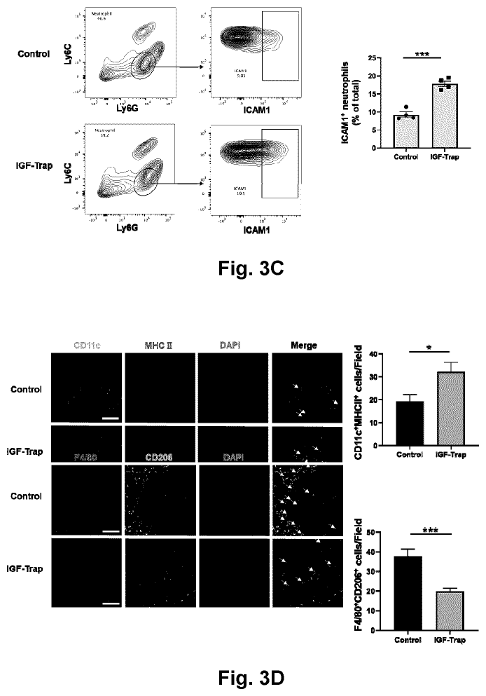

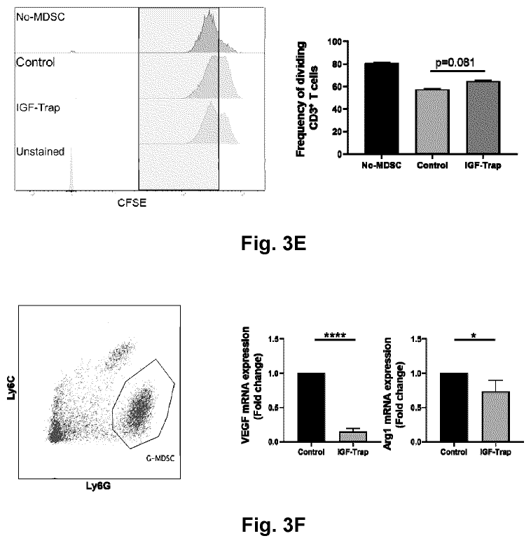

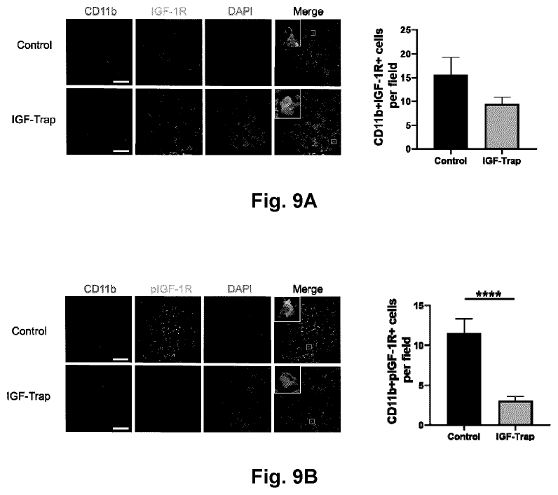

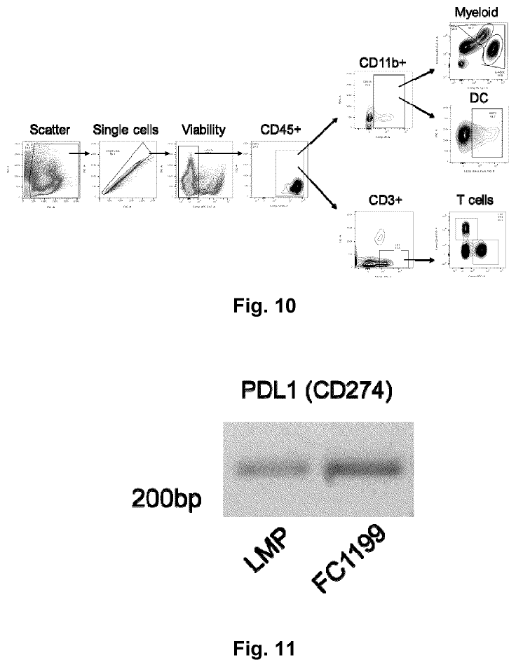

The IGF-Trap altered the immunosuppressive landscape associated with PDAC liver metastases. Having identified global changes in the liver TIME in IGF-Trap treated mice, it was sought to identify specific immune cell subtypes whose recruitment was affected by this treatment. Flow cytometry (FC) and immunofluorescence microscopy (IF) were used to compare the immune cell infiltrates in mice treated, or not, for 2 weeks with the IGF-Trap following the intrasplenic/portal inoculation of LMP cells. It was confirmed that IGF-IR activation levels were reduced in CD11b+ myeloid cells by IHC performed on liver sections from the treated mice (FIGS. 9A and 9B). Consistent with the data obtained with the digital profiler, it was found that the accumulation of CD11b+Ly6C+Ly6G+ myeloid cells, particularly (CD11b+Ly6C^{mid}Ly6G^{high}) G-MDSC was markedly reduced in the livers of IGF-Trap treated mice, as compared to controls (FIGS. 3A and 10). It was also found in the IGF-Trap treated mice an increased accumulation of (CD11b+CD11c+) DC that was accompanied by a 35% increase in MHCII expression in these cells (FIG. 3B), indicative of their increased activation levels in the treated mice. Consistent with previous findings in a colon carcinoma metastasis model (Rayes et al., 2018), it was also found a significant increase in the proportion of ICAM-1+ (CD11b+Ly6G^{high}Ly6C^{low}) neutrophils in the treated mice, indicative of reduced N2 polarization (FIG. 3C). Taken together, these results suggested that IGF-Trap mediated blockade of IGF-IR signaling resulted in an altered and less immunosuppressive TIME in the liver, with an enhanced potential to mount an anti-tumor immune response. This conclusion was further supported by fluorescence microscopy performed on liver cryostat sections obtained from tumor-bearing mice. Namely, a marked increase in CD11c+MHCII+ DC was observed around metastatic foci in IGF-Trap treated mice (FIG. 3D top), as compared to controls. Moreover, in both control and IGF-Trap treated mice, we detected F4/80+CD206+ macrophages around the metastases, indicative of the accumulation of M2 polarized TAM in the livers. However, the number of these TAM was significantly reduced (47.3%) in mice treated with the IGF-Trap (FIG. 3D bottom). Finally the immunosuppressive functions of tumor-infiltrating MDSC was analyzed using a T cell suppression assay and found that suppression of CD3+ T cell proliferation showed a decreased trend (p=0.08) when these cells were incubated with G-MDSC derived from IGF-Trap treated mice, as compared to control MDSC (FIG. 3E). Moreover, when CD11b+LY6Ghigh cells were isolated using FACS sorting and their RNA analyzed by qPCR, cells isolated from the IGF-Trap treated mice exhibited a significant reduction in VEGF and Arg1 expression (FIG. 3F), consistent with reduced immunosuppressive potency.

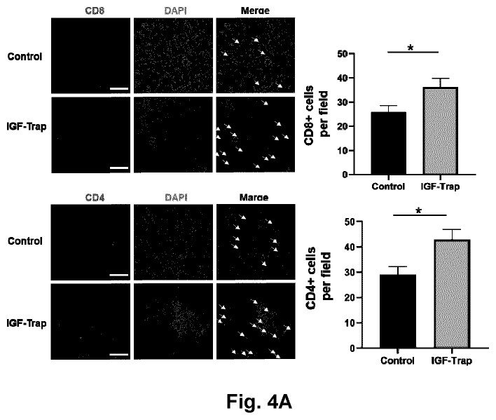

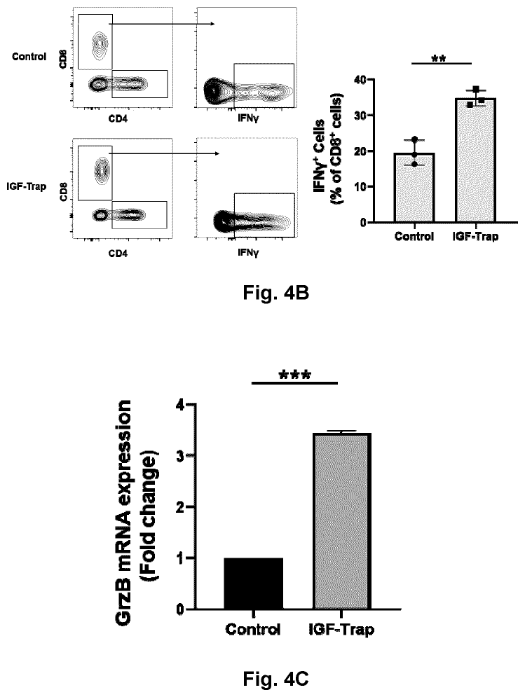

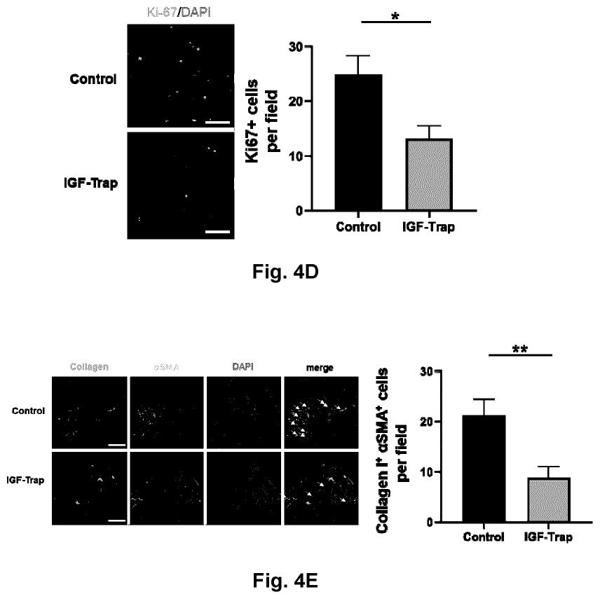

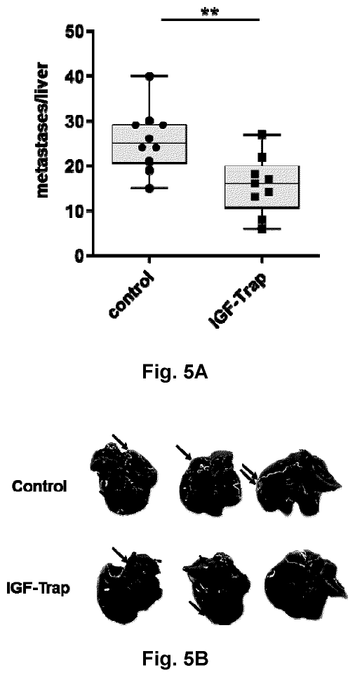

Taken together, these results identified in IGF-Trap treated mice a significant shift in the TIME of PDAC liver metastases from a predominantly immunosuppressive and pro-metastatic TME to one that is more conducive to anti-tumor immune responses. To determine whether this reduced accumulation and functional potency of immunosuppressive cells was, in fact, associated with increased accumulation in the liver of CD4+ and CD8+ T cells, an IHC analysis was performed on cryostat sections derived from tumor-inoculated mice and found increased accumulation of these T cells in the IGF-Trap treated mice (FIG. 4A).

To determine whether IGF-Trap treatment affected T cell activation and cytokine production in the liver, immune cells from the livers of LMP-inoculated mice were isolated and IFN-γ production levels was measured following stimulation of the cells with PMA and ionomycin. Flow cytometric analyses revealed increased IFN-γ levels in CD3+CD8+ T cells derived from the IGF-Trap treated mice (FIG. 4B), suggesting increased functional CTL activity. This was also confirmed when GrzB expression levels were measured by qPCR in CD3+CD8+ T cells isolated by FACS sorting from similarly treated mice, revealing increased GrzB expression levels in T cells isolated from IGF-Trap treated mice (FIG. 4C). Finally, this decrease in the accumulation and function of immunosuppressive cells coupled with increased CTL activity was associated with a reduction in Ki67+ tumor cells within metastatic foci, suggesting that the number of proliferating tumor cells was markedly reduced (FIG. 4D). Collectively, these data strongly suggested that IGF-Trap treatment caused a profound change in the, otherwise, immune-tolerant and pro-metastatic microenvironment associated with metastatic expansion in this organ.

Treatment with the IGF-Trap impairs HSC activation. Hepatic stellate cells (HSC) are activated during the early stages of liver metastasis and play a key role in the induction of a pro-metastatic microenvironment in this organ. Normally quiescent within the space of Disse, they are activated in response to factors released by innate immune cells that are recruited to sites of tumor invasion, differentiate into myofibroblast-like cells and characteristically express the myofibroblastic cell surface markers α-smooth muscle actin (α-SMA) and desmin and produce type I collagen, as well as immunoregulatory chemokines. Previously it was shown that IGF-I plays a role in HSC activation in the pro-inflammatory microenvironment induced by tumor cell entry into the liver (Fernandez et al., 2019). To investigate the effect of the IGF-Trap on HSC activation following injection of LMP cells, BL6129-Col-GFP mice were used, in which type I collagen (Coll) is genetically GFP-tagged and activated HSC can be identified based on co-expression of GFP-Coll and α-SMA. Consistent with previous findings, it was found that treatment of these mice with the IGF-Trap significantly reduced HSC activation in response to the metastatic cells (FIG. 4E).

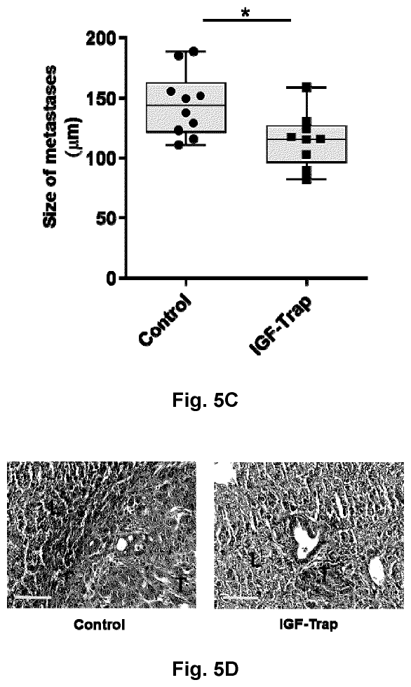

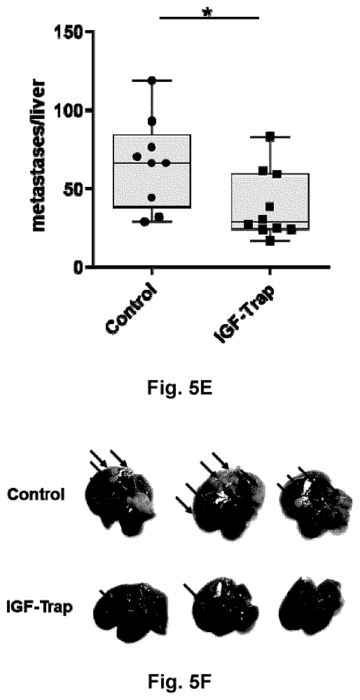

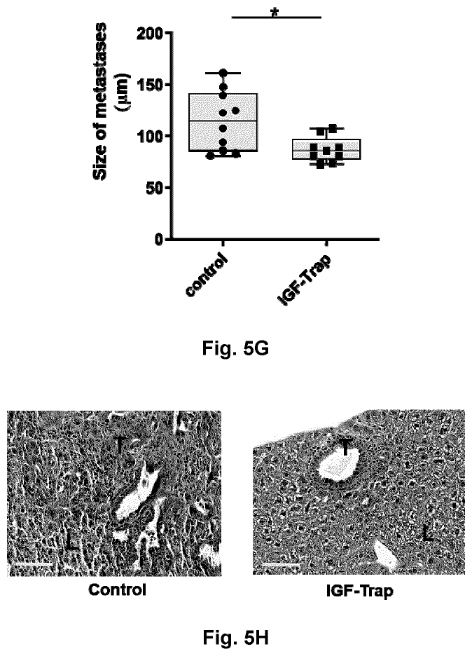

The IGF-Trap inhibited the growth of experimental PDAC liver metastases. Having previously observed marked differences in the immunosuppressive landscape within the TIME of liver metastases in mice treated with the IGF-Trap, it was analyzed how these changes affected metastatic expansion following injection of LMP cells via the intrasplenic/portal route to generate experimental liver metastases. In a previous study, a sexual dimorphism was reported in the control of the TIME of liver metastases (as reported in Milette et al., 2019), the present experiments were performed in age-matched male and female mice to rule out sex-specific effects. In both sexes, a significant reduction in the numbers and sizes of liver metastases was observed (FIG. 5A-H), as compared to treated controls, suggesting that the changes in the TIME impacted metastatic expansion in the livers of the treated mice, regardless of their sex.

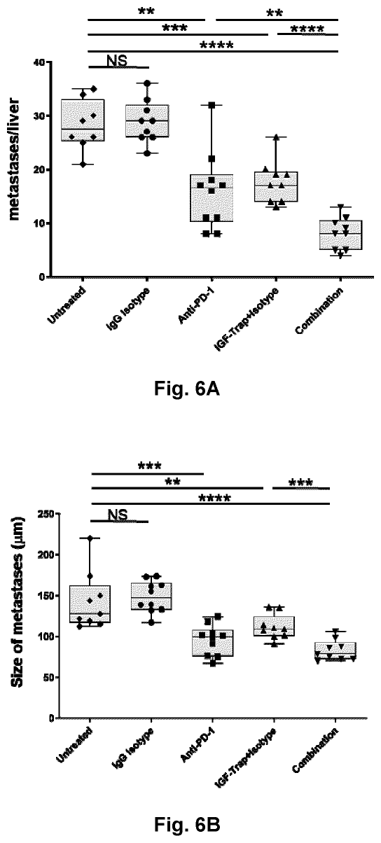

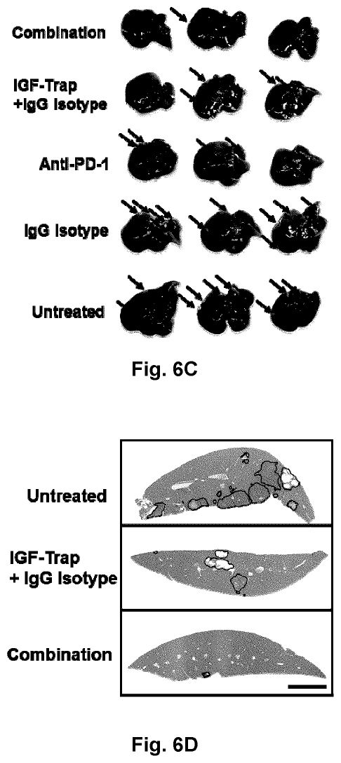

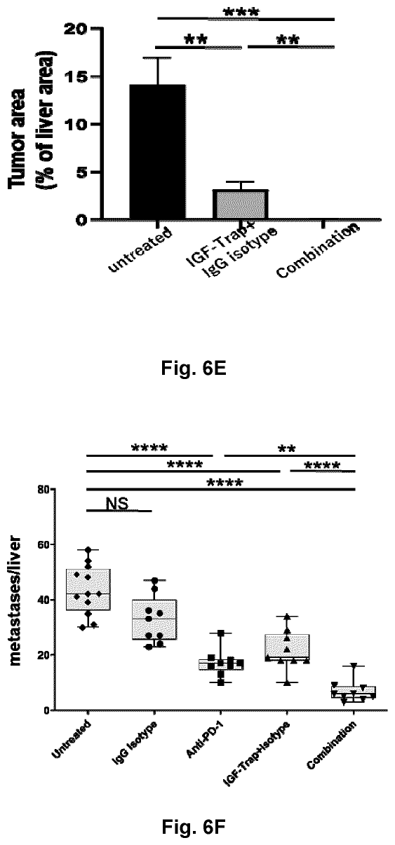

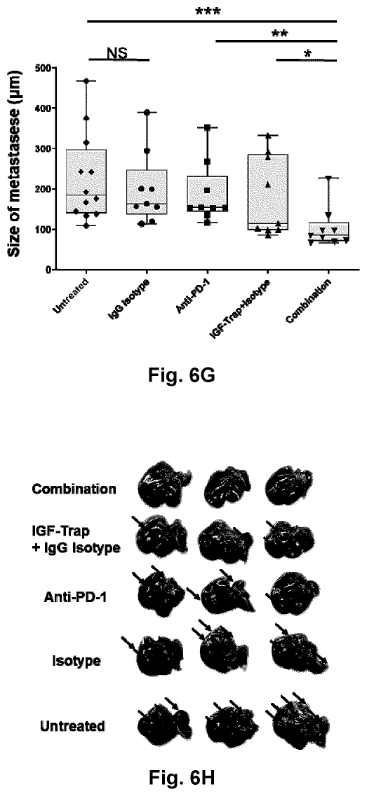

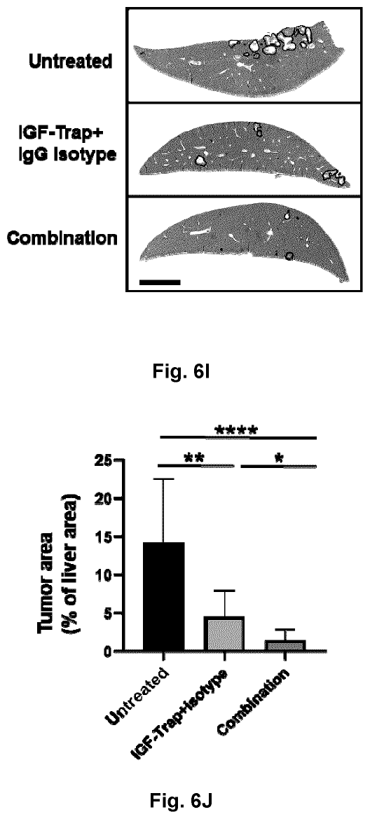

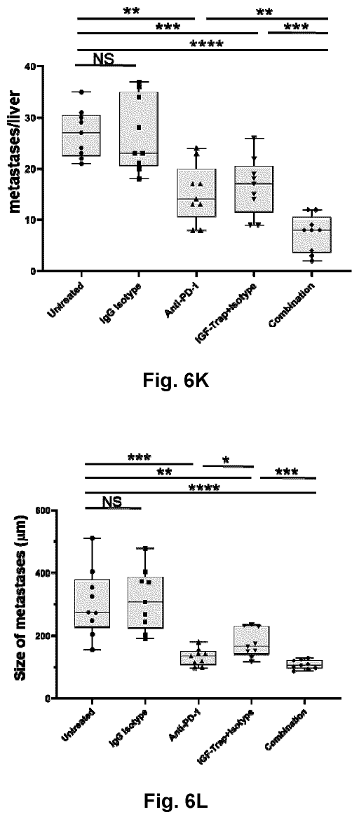

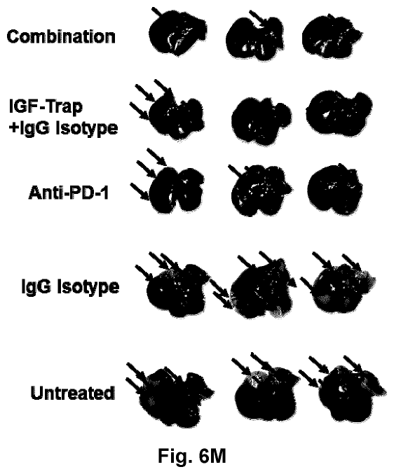

The IGF-Trap and immunotherapy reciprocally enhance their inhibitory effects on liver metastasis. The failure of PDAC patients to respond to immunotherapy is thought to be due, at least in part, to immunosuppressive cells such as polarized TAM and MDSC that infiltrate the primary tumors. While the TIME associated with PDAC liver metastases has not been extensively explored (partially because surgical resections of PDAC liver metastases are rare), it was recently documented the accumulation of immunosuppressive cells such as G-MDSC and Mo-MDSC in the livers of mice bearing spontaneous or experimental LMP metastases (Milette et al., 2019). The data above have clearly shown that IGF-Trap treatment caused a reduction in the accumulation and/or polarization of several immunosuppressive cell types in the livers, and this was associated with a reduction in the metastatic burden in these mice. Intriguingly, the increased accumulation of T cells in the treated mice was also associated with increased expression of PD-1 (FIGS. 2B and 8A-8B). It was confirmed that the expression of PD-L1 in LMP cells as well as in a second PDAC cell line the FC1199 cells syngeneic to C57BI/6 mice (FIG. 11). It was therefore determined whether, combining a PD-1 inhibitor with the IGF-Trap will have a reciprocal enhancing effect, potentiating anti-tumor immunity in this organ, and further reducing the metastatic burden. Mice inoculated with LMP cells via the intrasplenic/portal route were treated on alternate days with 5 mg/kg IGF-Trap or 10 mg/kg of an anti-murine PD-1 antibody (or a non-immune IgG isotype as control) for a total of 5 and 3 injections, respectively over a period of 15 days. Liver metastases were then enumerated 21 days post tumor injection and compared to mice injected with each of these inhibitors alone. Treatment with the IGF-Trap reduced liver metastasis when used alone and the addition of the IgG isotype control to the IGF-Trap had no effect on the metastatic burden. Treatment with the anti PD-1 antibody alone also reduced the number of liver metastases, suggesting that blockade of this immune checkpoint enabled an anti-tumor immune response in these mice. These tumor-inhibitory effects were markedly enhanced, when the two treatments were combined and this was observed in both male (FIGS. 6A-E) and female (FIGS. 6F-J) mice, and reflected in further reductions in both the median number and mean size of the metastases, revealing a synergistic inhibitory effect on the growth of liver metastases when the two drugs were combined. This marked effect of the combinatorial immunotherapy on liver metastases was not specific to the strain or the LMP model, as similar effects were also observed when C57BI/6 mice were injected with the syngeneic PDAC FC1199 cells and treated in a similar manner (FIGS. 6K-M).

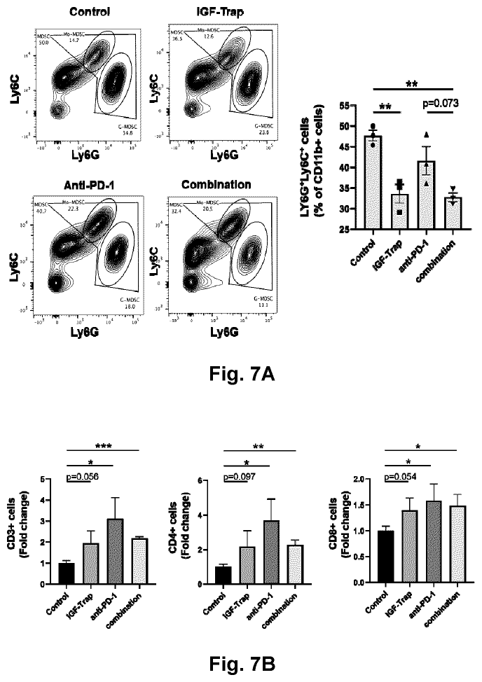

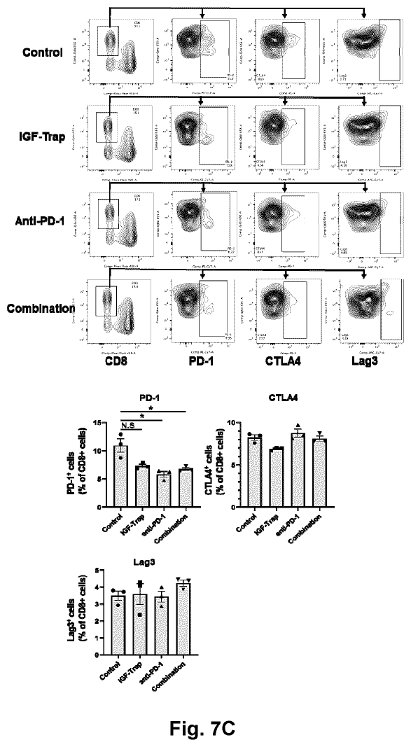

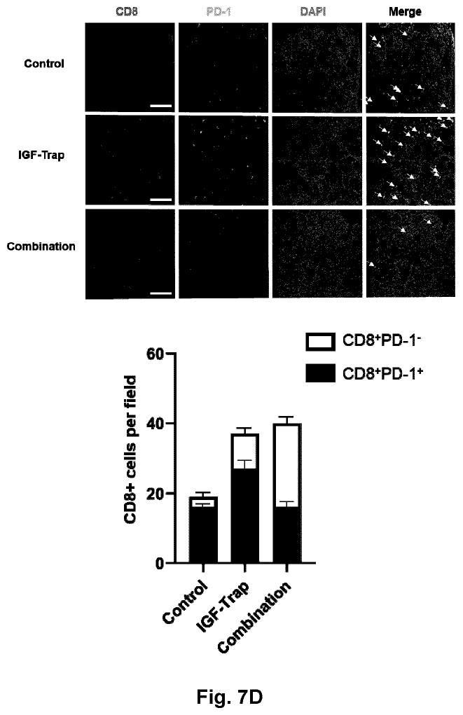

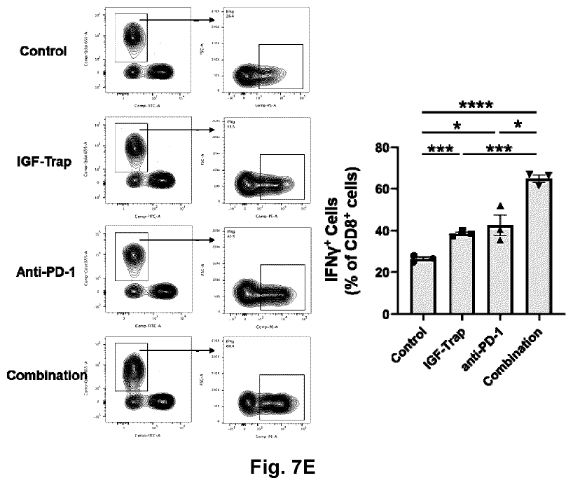

To elucidate the mechanism of action of the combinatorial treatment, the immune cell infiltrate was compared in mice injected with LMP cells treated with each of the inhibitors alone or with the combination, using FC and IF. Tumor-injected mice were treated as indicated (FIG. 7) and their livers analyzed 14 days post tumor inoculationA significant decrease in MDSC accumulation was observed in IGF-Trap treated mice, as well as in mice that received the combination therapy, as compared to control mice or mice treated with the anti PD-1 antibody, and this was particularly evident for the accumulation Ly6Ghigh G-MDSC (FIG. 7A). In mice treated with anti-PD-1 antibodies (alone or in combination with IGF-Trap), a significant increase was observed in the accumulation of both CD4+ and CD8+ T cells and a corresponding decrease in PD-1 expression on CD8+ cells and this was confirmed by both FC and IF. However, no change was observed in the expression of CTLA-4 or Lag3 on these cells (FIG. 7B-D). Finally, FC revealed a marked increase in IFN-γ producing CD8+ T cells in mice treated with the combinatorial therapy as compared to single agent-treated mice, indicating a further potentiation of a CTL response in these mice (FIG. 7E).

Despite recent successes of single-agent immunotherapy in the treatment of several highly aggressive malignancies, the majority of cancers, including PDAC remain unresponsive. This, despite evidence of T cell-activating tumor antigens on PDAC cells. Among several factors that may contribute to this failure to respond are the production of immunosuppressive IL-10 and TGF-β by PDAC cells and the recruitment to the tumor site of immunosuppressive cells such as MDSC and M2 TAM. This may be particularly true in the liver, where recruitment of bone marrow derived myeloid cells to form pre-metastatic niches has been documented and this is accelerated by tumor cell entry into the liver. Thus, combination therapies that can activate cytotoxic T cells while also targeting immunosuppressive cells in the TME hold much promise in the treatment of this disease. In this study, we show for the first time, that targeting the IGF axis alters the TIME of PDAC liver metastases, reducing the recruitment and activity of several immunosuppressive cell types and resulting in increased T cell accumulation in the liver. Furthermore, this treatment enhanced the anti-metastatic effect of a PD-1 inhibitor by potentiating a T

Intriguingly, a difference in the growth of the local, orthotopically implanted pancreatic tumors in mice treated with the IGF-Trap was not observed, although the growth of liver metastases in the same animals was inhibited. It was previously shown that the TIME in the pancreas and liver are distinct. While in the liver, a high proportion of Mo-MDSC was found, this population was absent in the pancreas and the G-MDSC in the two organs were differentially regulated. In the livers of mice treated with the IGF-Trap, a marked reduction in both Mo-MDSC and M2 macrophages was observed. The lack of effect on tumor growth in the pancreas may therefore be due, at least partially, to the absence of this population at this site. Moreover, as previously shown, the liver is the main site of IGF-Trap accumulation (Vaniotis et al., 2018), and IGF-Trap bioavailability within the pancreatic tumors may be further limited by the local stromal barrier, thus differences in the local concentrations of the IGF-Trap in the two organs may contribute to the differential effects.

It was shown here that the IGF-Trap treatment profoundly altered the immune landscape of liver metastases, affecting a multiplicity of immune cell types. While IGF-IR is widely expressed on innate and adaptive immune cells, and IGF-IR blockade could therefore, potentially, affect the recruitment and/or function of each of these cell types, it is also possible that this broad effect may be due to transcriptional regulation of a central factor that could otherwise induce a state of immunosuppression in the liver. It was previously shown that treatment of bone marrow-derived CD11b+Ly6G+ cells with IGF-I upregulated the expression of both TGF-β1 and VEGF, and as shown in the present example (FIGS. 2 and 8A and 8B), IGF-Trap treatment significantly reduced TGF-β1 levels in the TME of liver metastases, at both early and late stages of expansion. This could account for the overall reduction in the accumulation of tumor-associated immunosuppressive cells such as MDSC, M2 macrophages and N2 neutrophils that are all regulated by this immunosuppressive factor. Furthermore, because the recruitment of neutrophils and monocytes are early events in the mobilization of innate immune cells into the microenvironment of liver metastases, and this sets in motion later events such as activation of hepatic stellate cells that are an additional source of TGF-β, a blockade of IGF-IR signaling in these cells could initiate a chain reactions leading to a general decrease in the recruitment of immunosuppressive cells and increased accumulation of CTL. IGF-IR signaling blockade may, in addition, alter the production of cytokines such as TGF-β and VEGF by the PDAC cells, further reducing the pro-metastatic conditions in the liver. In this context, it should also be noted that in mice treated with the IGF-Trap, a reduction in activated IGF-IR levels in CD11b+ cells infiltrating the metastatic foci was observed, as well as in the tumor cells. While it was previously shown that IGF-Trap treatment can also alter the liver ME in a tumor-free model (Fernandez et al., 2017), it cannot entirely rule out the possibility that in the present model, a direct effect of the IGF-Trap on tumor cell proliferation, also contributed to reduced metastatic expansion.

The results presented herein add to the growing body of evidence that immunotherapy can be rendered more effective when combined with strategies that target the TME in PDAC and other advanced solid and treatment-refractory tumors. Some combinations with targeted therapy have, in fact, already advanced to clinical trials. The prognostic value of PD-1/PD-L1 expression together with infiltration of CD8+ lymphocytes and Treg in 145 surgical PDAC resections was found for both PD-1+ and CD8+ T cell infiltrates were independent prognostic markers in patients treated with adjuvant chemotherapy, predicting a better outcome. Moreover, a genomic analysis recently identified a PDAC subtype with increased activation of CD8+ T cells and overexpression of CTLA-4 and PD-1 within surgical resections that corresponded to higher frequency of somatic mutations and tumor-specific neoantigens, suggesting that patient stratification based on mutational signatures may identify a patient subpopulation particularly sensitive to immunotherapy. Collectively, these data identify PDAC as a malignancy that may be highly responsive to immune-based therapy under optimizing conditions. In the model presented herein, a significant effect on liver metastases was found when mice were treated with a single checkpoint inhibitor, namely anti PD-1 antibodies, despite an unaltered expression of both CTLA-4 and Lag3 on CD8+ T cells. This suggests that the effect of immunotherapy, in this model, may potentially be even further optimized by combining antibodies to several immune checkpoints with IGF-Trap treatment, and this may also be required in the clinical management of this disease.

Liver metastases were identified as one of several factors predictive of poor response to immunotherapy. This could be a contributing factor to the resistance of PDAC to immunotherapy, as a large proportion of PDAC patients already harbor hepatic metastases at the time of diagnosis or relapse with liver metastases following surgical excision of the primary tumor. The data, taken together with other findings, suggest that for patients with resectable primary PDAC tumors that are free of liver metastases at the time of diagnosis, combining immunotherapy with targeted anti-IGF-IR therapy could markedly improve treatment outcome.

Prolonged Survival of Mice Treated with the Combinatorial IGF-Trap/Anti PD-1 Therapy.

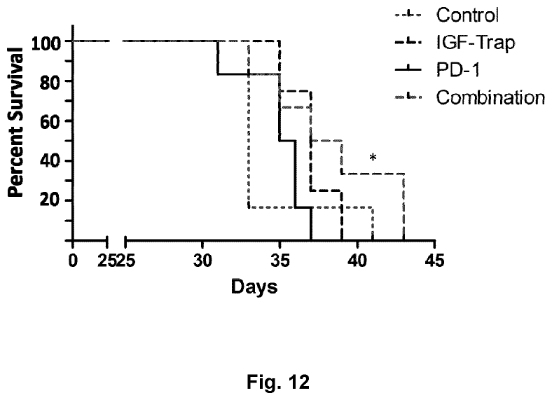

Experimental liver metastases were generated by intrasplenic/portal injection of 1×10⁵ LMP tumor cells into syngeneic female B6.129 F1 mice (5 mice per group, FIG. 12). Treatment with 5 mg/kg IGF-Trap (or vehicle) i.v. was initiated 1 day post tumor inoculation and continued twice weekly. Treatment with 10 mg/kg anti PD-1 antibody i.p was administered on alternate days as described in the legend to FIG. 6. Treatments continued for a total of 5 weeks or until animals were moribund and euthanized. Shown is a Kaplan Meier plot for each of the treatment groups. *p<0.05 as assessed by the Gehan-Breslow-Wilcoxon Test.

While the invention has been described in connection with specific embodiments thereof, it will be understood that the scope of the claims should not be limited by the preferred embodiments set forth in the examples, but should be given the broadest interpretation consistent with the description as a whole.

### REFERENCES

Fernandez M C, Rayes R, Ham B, Wang N, Bourdeau F, Milette S, et al. The type I insulin-like growth factor regulates the liver stromal response to metastatic colon carcinoma cells. Oncotarget. 2017; 8(32):52281-93.
Frese K K, Tuveson D A. Maximizing mouse cancer models. Nat Rev Cancer. 2007; 7(9):645-58.
Ham B, Wang N, D'Costa Z, Fernandez M C, Bourdeau F, Auguste P, et al. TNF Receptor-2 Facilitates an Immunosuppressive Microenvironment in the Liver to Promote the Colonization and Growth of Hepatic Metastases. Cancer Res. 2015; 75(24):5235-47.
Hingorani S R, Wang L, Multani A S, Combs C, Deramaudt T B, Hruban R H, et al. Trp53R172H and KrasG12D cooperate to promote chromosomal instability and widely metastatic pancreatic ductal adenocarcinoma in mice. Cancer Cell. 2005; 7(5):469-83.
Jiang Y J, Lee C L, Wang Q, Zhou Z W, Yang F, Jin C, et al. Establishment of an orthotopic pancreatic cancer mouse model: cells suspended and injected in Matrigel. World J Gastroenterol. 2014:20(28):9476-85.
Milette S, Hashimoto M, Perrino S, Qi S, Chen M, Ham B, et al. Sexual dimorphism and the role of estrogen in the immune microenvironment of liver metastases. Nat Commun. 2019; 10(1):5745.
Rayes R F, Milette S, Fernandez M C, Ham B, Wang N, Bourdeau F, et al. Loss of neutrophil polarization in colon carcinoma liver metastases of mice with an inducible, liver-specific IGF-I deficiency. Oncotarget. 2018; 9(21):15691-704.
Tseng W W, Winer D, Kenkel J A, Choi O, Shain A H, Pollack J R, et al. Development of an orthotopic model of invasive pancreatic cancer in an immunocompetent murine host. Clin Cancer Res. 2010; 16(14):3684-95.
Vaniotis G, Moffett S, Sulea T, Wang N, Elahi S M, Lessard E, et al. Enhanced anti-metastatic bioactivity of an IGF-TRAP re-engineered to improve physicochemical properties. Sci Rep. 2018; 8(1):17361.
Wang N, Rayes R F, Elahi S M, Lu Y, Hancock M A, Massie B, et al. The IGF-Trap: Novel Inhibitor of Carcinoma Growth and Metastasis. Mol Cancer Ther. 2015; 14(4):982-93.

1. A method for alleviating a symptom of or treating a cancer in a subject in need thereof, the method comprising administrating an effective amount of an antagonist of a type I insulin-like growth factor receptor (IGF-1R) prior to, concomitantly and/or after having administered an effective amount of an immune response activating agent to the subject so as to alleviate the symptom or treat the cancer.

2. (canceled)

3. The method of claim 1, wherein the antagonist of the IGF-1R comprises a soluble IGF receptor (IGF-R).

4. The method of claim 3, wherein the antagonist of the IGF-R is a chimeric protein comprising the soluble IGF-1 receptor as a first moiety and a human Fc as a second moiety.

5. The method of claim 4, wherein the antagonist of the IGF-1R comprises the amino acid sequence of SEQ ID NO: 6 or 8, a variant of the amino acid sequence of SEQ ID NO: 6 or 8 or a fragment of the amino acid sequence of SEQ ID NO: 6 or 8.

6. The method of claim 1, wherein the immune response activating agent comprises an antagonistic antibody.

7. (canceled)

8. The method of claim 6, wherein the anti-cancer immune stimulating agent comprises an anti-cytotoxic T-lymphocyte associated protein 4 (CTLA-4) antibody, an anti-programmed cell death 1 (PD-1) antibody, an anti-programmed cell death 1 ligand (PD-L1) antibody, an anti-programmed cell death 2 ligand (PD-L2) antibody, an anti-T cell immunoglobulin and mucin domain 3 (TIM-3) antibody and/or an anti-lymphocyte activation gene-3 (LAG-3) antibody.

9. (canceled)

10. The method of claim 6, wherein the immune response activating agent comprises an anti-TNF receptor superfamily member 9 (TNFRSF9) antibody, an anti-TNF receptor superfamily member 4 (TNFRSF4) antibody and/or an anti-TNF receptor superfamily member 18 (TNFRSF18) antibody.

11-16. (canceled)

17. A method of reducing the immune suppression and increasing the immune cytotoxicity in a tissue in need thereof, the method comprising contacting an antagonist of a type 1 insulin growth factor receptor (IGF-1R) with the tissue so as to reduce the immune suppression and increase the immune cytotoxicity when compared to a control tissue that was not contacted with the antagonist of the IGF-1R.

18. The method of claim 17 for reducing myeloid derived suppressor cells, immunosuppressive (N2) neutrophils and/or anti-inflammatory immunosuppressive (M2) tumor-associate macrophages in the tissue.

19. The method of claim 17 for increasing and activating dendritic cells and/or increasing pro-inflammatory (N1) neutrophils in the tissue.

20-26. (canceled)

27. The method of claim 17 for increasing, in the tissue, when compared to the control tissue:
CD11b+, CD11c+ and MHCII+ immune-accessory cells;
CD11c+ and MHCII+ immune response cells;
ICAM-1+ immune cells;
CD4+ immune cells;
CD8+ cells; and/or
CD68+ cells.

28. (canceled)

29. The method of claim 17 for decreasing, in the tissue, when compared to the control tissue:
CD11b+, Ly6G+ and Ly6C+ immunosuppressive cells;
CD163+ immune cells; and/or
CD206+ immune cells.

30. The method of claim 17 for decreasing, in the tissue, when compared to the control tissue:
TGF-β;
collagen I; and/or
α-smooth muscle actin expressing cells.

31. The method of claim 17 for increasing, in the tissue when compared to a control tissue:
IFN-γ; and/or
granzyme B.

32-34. (canceled)

35. A combination therapy for alleviating a symptom of or treating a cancer in a subject in need thereof, said combination therapy comprising an effective amount of an antagonist of a type I insulin-like growth factor receptor (IGF-1R) and an effective amount of an immune response activating agent.

36. The combination therapy of claim 35, wherein the antagonist of the IGF-1R comprises a soluble IGF receptor (IGF-R).

37. The combination therapy of claim 36, wherein the antagonist of the IGF-R is a chimeric protein comprising the soluble IGF-1 receptor as a first moiety and a human Fc as a second moiety.

38. The combination therapy of claim 37, wherein the antagonist of the IGF-1R comprises the amino acid sequence of SEQ ID NO: 6 or 8, a variant of the amino acid sequence of SEQ ID NO: 6 or 8 or a fragment of the amino acid sequence of SEQ ID NO: 6 or 8.

39. The combination therapy of any one of claims 35-38, wherein the immune response activating agent comprises an antagonistic antibody.

40. (canceled)

41. The combination therapy of claim 39, wherein the anti-cancer immune stimulating agent comprises an anti-cytotoxic T-lymphocyte associated protein 4 (CTLA-4) antibody, an anti-programmed cell death 1 (PD-1) antibody, an anti-programmed cell death 1 ligand (PD-L1) antibody, an anti-programmed cell death 2 ligand (PD-L2) antibody, an anti-T cell immunoglobulin and mucin domain 3 (TIM-3) antibody and/or an anti-lymphocyte activation gene-3 (LAG-3) antibody.

42. (canceled)

43. The combination therapy of claim 39, wherein the immune response activating agent comprises an anti-TNF receptor superfamily member 9 (TNFRSF9) antibody, an anti-TNF receptor superfamily member 4 (TNFRSF4) antibody and/or an anti-TNF receptor superfamily member 18 (TNFRSF18) antibody.

44-67. (canceled)


SEQUENCE LISTING (24 sequences)

SEQ ID NO: 1 - protein, 1367 aa, Homo sapiens
MKSGSGGGSPTSLWGLLFLSAALSLWPTSGEICGPGIDIRNDYQQLKRLENCTVIEGYLHILLISKAEDYRSYRFPKLTVITEYLLLFRVAGLESLGDLFPNLTVIRGWKLFYNYALVIFEMTNLKDIGLYNLRNITRGAIRIEKNADLCYLSTVDWSLILDAVSNNYIVGNKPPKECGDLCPGTMEEKPMCEKTTINNEYNYRCWTTNRCQKMCPSTCGKRACTENNECCHPECLGSCSAPDNDTACVACRHYYYAGVCVPACPPNTYRFEGWRCVDRDFCANILSAESSDSEGFVIHDGECMQECPSGFIRNGSQSMYCIPCEGPCPKVCEEEKKTKTIDSVTSAQMLQGCTIFKGNLLINIRRGNNIASELENFMGLIEVVTGYVKIRHSHALVSLSFLKNLRLILGEEQLEGNYSFYVLDNQNLQQLWDWDHRNLTIKAGKMYFAFNPKLCVSEIYRMEEVTGTKGRQSKGDINTRNNGERASCESDVLHFTSTTTSKNRIIITWHRYRPPDYRDLISFTVYYKEAPFKNVTEYDGQDACGSNSWNMVDVDLPPNKDVEPGILLHGLKPWTQYAVYVKAVTLTMVENDHIRGAKSEILYIRTNASVPSIPLDVLSASNSSSQLIVKWNPPSLPNGNLSYYIVRWQRQPQDGYLYRHNYCSKDKIPIRKYADGTIDIEEVTENPKTEVCGGEKGPCCACPKTEAEKQAEKEEAEYRKVFENFLHNSIFVPRPERKRRDVMQVANTTMSSRSRNTTAADTYNITDPEELETEYPFFESRVDNKERTVISNLRPFTLYRIDIHSCNHEAEKLGCSASNFVFARTMPAEGADDIPGPVTWEPRPENSIFLKWPEPENPNGLILMYEIKYGSQVEDQRECVSRQEYRKYGGAKLNRLNPGNYTARIQATSLSGNGSWTDPVFFYVQAKTGYENFIHLIIALPVAVLLIVGGLVIMLYVFHRKRNNSRLGNGVLYASVNPEYFSAADVYVPDEWEVAREKITMSRELGQGSFGMVYEGVAKGVVKDEPETRVAIKTVNEAASMRERIEFLNEASVMKEFNCHHVVRLLGVVSQGQPTLVIMELMTRGDLKSYLRSLRPEMENNPVLAPPSLSKMIQMAGEIADGMAYLNANKFVHRDLAARNCMVAEDFTVKIGDFGMTRDIYETDYYRKGGKGLLPVRWMSPESLKDGVFTTYSDVWSFGVVLWEIATLAEQPYQGLSNEQVLRFVMEGGLLDKPDNCPDMLFELMRMCWQYNPKMRPSFLEIISSIKEEMEPGFREVSFYYSEENKLPEPEELDLEPENMESVPLDPSASSSSLPLPDRHSGHKAENGPGPGVLVLRASFDERQPYAHMNGGRKNERALPLPQSSTC

SEQ ID NO: 2 - protein, 933 aa, Artificial Sequence
  sIGF-1R933
MKSGSGGGSPTSLWGLLFLSAALSLWPTSGEICGPGIDIRNDYQQLKRLENCTVIEGYLHILLISKAEDYRSYRFPKLTVITEYLLLFRVAGLESLGDLFPNLTVIRGWKLFYNYALVIFEMTNLKDIGLYNLRNITRGAIRIEKNADLCYLSTVDWSLILDAVSNNYIVGNKPPKECGDLCPGTMEEKPMCEKTTINNEYNYRCWTTNRCQKMCPSTCGKRACTENNECCHPECLGSCSAPDNDTACVACRHYYYAGVCVPACPPNTYRFEGWRCVDRDFCANILSAESSDSEGFVIHDGECMQECPSGFIRNGSQSMYCIPCEGPCPKVCEEEKKTKTIDSVTSAQMLQGCTIFKGNLLINIRRGNNIASELENFMGLIEVVTGYVKIRHSHALVSLSFLKNLRLILGEEQLEGNYSFYVLDNQNLQQLWDWDHRNLTIKAGKMYFAFNPKLCVSEIYRMEEVTGTKGRQSKGDINTRNNGERASCESDVLHFTSTTTSKNRIIITWHRYRPPDYRDLISFTVYYKEAPFKNVTEYDGQDACGSNSWNMVDVDLPPNKDVEPGILLHGLKPWTQYAVYVKAVTLTMVENDHIRGAKSEILYIRTNASVPSIPLDVLSASNSSSQLIVKWNPPSLPNGNLSYYIVRWQRQPQDGYLYRHNYCSKDKIPIRKYADGTIDIEEVTENPKTEVCGGEKGPCCACPKTEAEKQAEKEEAEYRKVFENFLHNSIFVPRPERKRRDVMQVANTTMSSRSRNTTAADTYNITDPEELETEYPFFESRVDNKERTVISNLRPFTLYRIDIHSCNHEAEKLGCSASNFVFARTMPAEGADDIPGPVTWEPRPENSIFLKWPEPENPNGLILMYEIKYGSQVEDQRECVSRQEYRKYGGAKLNRLNPGNYTARIQATSLSGNGSWTDPVFFYVQAKTGYENF

SEQ ID NO: 3 - protein, 933 aa, Artificial Sequence; identical to SEQ ID NO: 2
  IGF-1R

SEQ ID NO: 4 - protein, 1171 aa, Artificial Sequence
  h-sIGF1R-Fc
MKSGSGGGSPTSLWGLLFLSAALSLWPTSGEICGPGIDIRNDYQQLKRLENCTVIEGYLHILLISKAEDYRSYRFPKLTVITEYLLLFRVAGLESLGDLFPNLTVIRGWKLFYNYALVIFEMTNLKDIGLYNLRNITRGAIRIEKNADLCYLSTVDWSLILDAVSNNYIVGNKPPKECGDLCPGTMEEKPMCEKTTINNEYNYRCWTTNRCQKMCPSTCGKRACTENNECCHPECLGSCSAPDNDTACVACRHYYYAGVCVPACPPNTYRFEGWRCVDRDFCANILSAESSDSEGFVIHDGECMQECPSGFIRNGSQSMYCIPCEGPCPKVCEEEKKTKTIDSVTSAQMLQGCTIFKGNLLINIRRGNNIASELENFMGLIEVVTGYVKIRHSHALVSLSFLKNLRLILGEEQLEGNYSFYVLDNQNLQQLWDWDHRNLTIKAGKMYFAFNPKLCVSEIYRMEEVTGTKGRQSKGDINTRNNGERASCESDVLHFTSTTTSKNRIIITWHRYRPPDYRDLISFTVYYKEAPFKNVTEYDGQDACGSNSWNMVDVDLPPNKDVEPGILLHGLKPWTQYAVYVKAVTLTMVENDHIRGAKSEILYIRTNASVPSIPLDVLSASNSSSQLIVKWNPPSLPNGNLSYYIVRWQRQPQDGYLYRHNYCSKDKIPIRKYADGTIDIEEVTENPKTEVCGGEKGPCCACPKTEAEKQAEKEEAEYRKVFENFLHNSIFVPRPERKRRDVMQVANTTMSSRSRNTTAADTYNITDPEELETEYPFFESRVDNKERTVISNLRPFTLYRIDIHSCNHEAEKLGCSASNFVFARTMPAEGADDIPGPVTWEPRPENSIFLKWPEPENPNGLILMYEIKYGSQVEDQRECVSRQEYRKYGGAKLNRLNPGNYTARIQATSLSGNGSWTDPVFFYVQAKTGYENFEFDISSTMVRSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 1161 aa, Artificial Sequence
  sIGF1R-hFc-IgG2
MKSGSGGGSPTSLWGLLFLSAALSLWPTSGEICGPGIDIRNDYQQLKRLENCTVIEGYLHILLISKAEDYRSYRFPKLTVITEYLLLFRVAGLESLGDLFPNLTVIRGWKLFYNYALVIFEMTNLKDIGLYNLRNITRGAIRIEKNADLCYLSTVDWSLILDAVSNNYIVGNKPPKECGDLCPGTMEEKPMCEKTTINNEYNYRCWTTNRCQKMCPSTCGKRACTENNECCHPECLGSCSAPDNDTACVACRHYYYAGVCVPACPPNTYRFEGWRCVDRDFCANILSAESSDSEGFVIHDGECMQECPSGFIRNGSQSMYCIPCEGPCPKVCEEEKKTKTIDSVTSAQMLQGCTIFKGNLLINIRRGNNIASELENFMGLIEVVTGYVKIRHSHALVSLSFLKNLRLILGEEQLEGNYSFYVLDNQNLQQLWDWDHRNLTIKAGKMYFAFNPKLCVSEIYRMEEVTGTKGRQSKGDINTRNNGERASCESDVLHFTSTTTSKNRIIITWHRYRPPDYRDLISFTVYYKEAPFKNVTEYDGQDACGSNSWNMVDVDLPPNKDVEPGILLHGLKPWTQYAVYVKAVTLTMVENDHIRGAKSEILYIRTNASVPSIPLDVLSASNSSSQLIVKWNPPSLPNGNLSYYIVRWQRQPQDGYLYRHNYCSKDKIPIRKYADGTIDIEEVTENPKTEVCGGEKGPCCACPKTEAEKQAEKEEAEYRKVFENFLHNSIFVPRPERKRRDVMQVANTTMSSRSRNTTAADTYNITDPEELETEYPFFESRVDNKERTVISNLRPFTLYRIDIHSCNHEAEKLGCSASNFVFARTMPAEGADDIPGPVTWEPRPENSIFLKWPEPENPNGLILMYEIKYGSQVEDQRECVSRQEYRKYGGAKLNRLNPGNYTARIQATSLSGNGSWTDPVFFYVQAKTGYENFERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 6 - protein, 1171 aa, Artificial Sequence
  Chimeric polypeptide Mod#1
MKSGSGGGSPTSLWGLLFLSAALSLWPTSGEICGPGIDIRNDYQQLKRLENCTVIEGYLHILLISKAEDYRSYRFPKLTVITEYLLLFRVAGLESLGDLFPNLTVIRGWKLFYNYALVIFEMTNLKDIGLYNLRNITRGAIRIEKNADLCYLSTVDWSLILDAVSNNYIVGNKPPKECGDLCPGTMEEKPMCEKTTINNEYNYRCWTTNRCQKMCPSTCGKRACTENNECCHPECLGSCSAPDNDTACVACRHYYYAGVCVPACPPNTYRFEGWRCVDRDFCANILSAESSDSEGFVIHDGECMQECPSGFIRNGSQSMYCIPCEGPCPKVCEEEKKTKTIDSVTSAQMLQGCTIFKGNLLINIRRGNNIASELENFMGLIEVVTGYVKIRHSHALVSLSFLKNLRLILGEEQLEGNYSFYVLDNQNLQQLWDWDHRNLTIKAGKMYFAFNPKLCVSEIYRMEEVTGTKGRQSKGDINTRNNGERASCESDVLHFTSTTTSKNRIIITWHRYRPPDYRDLISFTVYYKEAPFKNVTEYDGQDACGSNSWNMVDVDLPPNKDVEPGILLHGLKPWTQYAVYVKAVTLTMVENDHIRGAKSEILYIRTNASVPSIPLDVLSASNSSSQLIVKWNPPSLPNGNLSYYIVRWQRQPQDGYLYRHNYCSKDKIPIRKYADGTIDIEEVTENPKTEVCGGEKGPCCACPKTEAEKQAEKEEAEYRKVFENFLHNSIFVPRPERKRRDVMQVANTTMSSRSRNTTAADTYNITDPEELETEYPFFESRVDNKERTVISNLRPFTLYRIDIHSCNHEAEKLGCSASNFVFARTMPAEGADDIPGPVTWEPRPENSIFLKWPEPENPNGLILMYEIKYGSQVEDQRECVSRQEYRKYGGAKLNRLNPGNYTARIQATSLSGNGSWTDPVFFYVQAKTGYENFEFDISSTMVRSDKTHTSPPSPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 1182 aa, Artificial Sequence
  Chimeric polypeptide Mod#2
MKSGSGGGSPTSLWGLLFLSAALSLWPTSGEICGPGIDIRNDYQQLKRLENCTVIEGYLHILLISKAEDYRSYRFPKLTVITEYLLLFRVAGLESLGDLFPNLTVIRGWKLFYNYALVIFEMTNLKDIGLYNLRNITRGAIRIEKNADLCYLSTVDWSLILDAVSNNYIVGNKPPKECGDLCPGTMEEKPMCEKTTINNEYNYRCWTTNRCQKMCPSTCGKRACTENNECCHPECLGSCSAPDNDTACVACRHYYYAGVCVPACPPNTYRFEGWRCVDRDFCANILSAESSDSEGFVIHDGECMQECPSGFIRNGSQSMYCIPCEGPCPKVCEEEKKTKTIDSVTSAQMLQGCTIFKGNLLINIRRGNNIASELENFMGLIEVVTGYVKIRHSHALVSLSFLKNLRLILGEEQLEGNYSFYVLDNQNLQQLWDWDHRNLTIKAGKMYFAFNPKLCVSEIYRMEEVTGTKGRQSKGDINTRNNGERASCESDVLHFTSTTTSKNRIIITWHRYRPPDYRDLISFTVYYKEAPFKNVTEYDGQDACGSNSWNMVDVDLPPNKDVEPGILLHGLKPWTQYAVYVKAVTLTMVENDHIRGAKSEILYIRTNASVPSIPLDVLSASNSSSQLIVKWNPPSLPNGNLSYYIVRWQRQPQDGYLYRHNYCSKDKIPIRKYADGTIDIEEVTENPKTEVCGGEKGPCCACPKTEAEKQAEKEEAEYRKVFENFLHNSIFVPRPERKRRDVMQVANTTMSSRSRNTTAADTYNITDPEELETEYPFFESRVDNKERTVISNLRPFTLYRIDIHSCNHEAEKLGCSASNFVFARTMPAEGADDIPGPVTWEPRPENSIFLKWPEPENPNGLILMYEIKYGSQVEDQRECVSRQEYRKYGGAKLNRLNPGNYTARIQATSLSGNGSWTDPVFFYVQAKTGYENFGGGGSGGGGSGGGGSGGGGSGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 1182 aa, Artificial Sequence [sequence omitted]

SEQ ID NO: 9 - protein, 1179 aa, Artificial Sequence
  Chimeric polypeptide Mod#4
MKSGSGGGSPTSLWGLLFLSAALSLWPTSGEICGPGIDIRNDYQQLKRLENCTVIEGYLHILLISKAEDYRSYRFPKLTVITEYLLLFRVAGLESLGDLFPNLTVIRGWKLFYNYALVIFEMTNLKDIGLYNLRNITRGAIRIEKNADLCYLSTVDWSLILDAVSNNYIVGNKPPKECGDLCPGTMEEKPMCEKTTINNEYNYRCWTTNRCQKMCPSTCGKRACTENNECCHPECLGSCSAPDNDTACVACRHYYYAGVCVPACPPNTYRFEGWRCVDRDFCANILSAESSDSEGFVIHDGECMQECPSGFIRNGSQSMYCIPCEGPCPKVCEEEKKTKTIDSVTSAQMLQGCTIFKGNLLINIRRGNNIASELENFMGLIEVVTGYVKIRHSHALVSLSFLKNLRLILGEEQLEGNYSFYVLDNQNLQQLWDWDHRNLTIKAGKMYFAFNPKLCVSEIYRMEEVTGTKGRQSKGDINTRNNGERASCESDVLHFTSTTTSKNRIIITWHRYRPPDYRDLISFTVYYKEAPFKNVTEYDGQDACGSNSWNMVDVDLPPNKDVEPGILLHGLKPWTQYAVYVKAVTLTMVENDHIRGAKSEILYIRTNASVPSIPLDVLSASNSSSQLIVKWNPPSLPNGNLSYYIVRWQRQPQDGYLYRHNYCSKDKIPIRKYADGTIDIEEVTENPKTEVCGGEKGPCCACPKTEAEKQAEKEEAEYRKVFENFLHNSIFVPRPERKRRDVMQVANTTMSSRSRNTTAADTYNITDPEELETEYPFFESRVDNKERTVISNLRPFTLYRIDIHSCNHEAEKLGCSASNFVFARTMPAEGADDIPGPVTWEPRPENSIFLKWPEPENPNGLILMYEIKYGSQVEDQRECVSRQEYRKYGGAKLNRLNPGNYTARIQATSLSGNGSWTDPVFFYVQAKTGYENFGGGGSGGGGSGGGGSGGGGSGGGGSGGCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 11 aa, Artificial Sequence
  Linker
EFDISSTMVRS

SEQ ID NO: 11 - protein, 22 aa, #Artificial Sequence
  Linker
GGGGSGGGGSGGGGSGGGGSGG

SEQ ID NO: 12 - protein, 27 aa, Artificial Sequence
  Linker
GGGGSGGGGSGGGGSGGGGSGGGGSGG

SEQ ID NO: 13 - protein, 227 aa, Artificial Sequence
  Fc portion
DKTHTSPPSPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 227 aa, Artificial Sequence
  Fc portion
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 227 aa, Artificial Sequence; identical to SEQ ID NO: 13
  Fc portion

SEQ ID NO: 16 - protein, 219 aa, Artificial Sequence
  Fc portion
CPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - DNA, 20 bp, Artificial Sequence
  Forward Arg1 primer
ggaagacagcagaggaggtg

SEQ ID NO: 18 - DNA, 20 bp, Artificial Sequence
  Reverse Arg1 primer
tatggttaccctcccgttga

SEQ ID NO: 19 - DNA, 20 bp, Artificial Sequence
  Forward VEGF primer
ttgagaccctggtggacatc

SEQ ID NO: 20 - DNA, 20 bp, Artificial Sequence
  Reverse VEGF primer
cacacaggacggcttgaaga

SEQ ID NO: 21 - DNA, 18 bp, Artificial Sequence
  Forward GrzB primer
cctcctgctactgctgac

SEQ ID NO: 22 - DNA, 19 bp, Artificial Sequence
  Reverse GrzB primer
gtcagcacaaagtcctctc

SEQ ID NO: 23 - DNA, 20 bp, Artificial Sequence
  Forward GAPDH primer
acccagaagactgtggatgg

SEQ ID NO: 24 - DNA, 20 bp, Artificial Sequence
  Reverse GAPDH primer
acccagaagactgtggatgg